(19) United States
(12) Patent Application Publication
(10) Pub. No.: US 2025/0186647 A1
(43) Pub. Date: Jun. 12, 2025

(54) SYSTEMS AND METHODS FOR NEUTRALIZING MATTER

(71) Applicant: EPIRUS, INC., Torrance, CA (US)

(72) Inventors: Harry Bourne Marr, Jr., Manhattan Beach, CA (US); Skyler Evan Granatir, Marina Del Rey, CA (US); William Griffin Dower, Torrance, CA (US)

(21) Appl. No.: 18/856,036

(22) PCT Filed: Apr. 7, 2023
(86) PCT No.: PCT/US2023/017931

§ 371 Date: Oct. 10, 2024

Related U.S. Application Data
(60) Provisional application No. 63/362,805, filed on Apr. 11, 2022.

(51) Int. Cl. A61L 9/18; A61L 2/08; A61L 2/24

(52) U.S. Cl. CPC A61L 9/18; A61L 2/08; A61L 2/24; A61L 2202/11; A61L 2202/14; A61L 2202/16; A61L 2209/111

(57) ABSTRACT

A radio frequency system is provided that can dynamically adjust or tune the emission of electromagnetic radiation such that the tuned electromagnetic radiation can destroy, neutralize, mitigate, and/or deactivate the viruses or bacteria and/or harmful organic materials present in an environment or any surface or any object without harming the human cells.

### TECHNICAL FIELD

This disclosure relates generally to sanitization, deactivation of harmful biological materials, neutralization of other materials, and therapeutic systems and methods.

### BACKGROUND

Some biological material, such as viruses and bacteria, can be detrimental to human health and economy. Most recently, for instance, a global pandemic caused by the SARS-COV-2 virus has resulted in thousands of deaths across the world and wreaked havoc on the global economy. The global pandemic has revealed the difficulty of economically mitigating harmful viruses and bacteria in large spaces. As another example, the presence of pollution and carbon dioxide poses significant health risks to people and animals alike.

### SUMMARY

This disclosure provides systems and methods that can deactivate, mitigate, destroy and/or neutralize materials present in an environment. Some examples of materials may include but is not limited to gases, viruses, bacteria, fungi, parasites, microbes, biological cells, proteins, spores, proteins, amino acids, prions, microbes, harmful organic materials, such as other harmful biological matter. The systems described herein are configured to use a radio frequency (RF) transmitter system (or RF device) configured to generate and/or emit electromagnetic radiation (EM) characterized by a frequency and/or power level to direct the EM radiation to destroy, neutralize, mitigate and/or deactivate one or more harmful materials in a volume. In various implementations, the frequency and/or the power level of the radiation can be selected to destroy, deactivate, neutralize and/or render harmful material without causing significant health risks to human cells and other useful biological matter. For example, the frequency of the EM radiation may be configured to resonate with a constituent of the target material, such as, for example, resonating with the frequency of capsid of the viruses, bacteria, and/or other harmful materials.

The systems described herein can be a handheld system or a system that has a mounting plate configured for mounting system to a surface. Various embodiments of the systems described herein can be configured to be disposed in a variety of environments including but not limited to residential buildings, hotels, office buildings, warehouses, manufacturing facilities, hospitals, surgical facilities, cruise ships, airplanes, military facilities, educational institutions, airports, and arenas. The systems described herein can be configured to be in a variety of size, shapes, weight, and power depending on the application and the requirements. For example, in some implementations, the system can be configured as a stand-alone unit that can be placed in a room and/or can be incorporated in a vent and/or can be plugged in a socket, etc. As another example, in some implementations, the system can be configured to have a slim profile capable of being disposed in doors and entryways. Yet another example, in various implementations, the systems can be configured to be handheld. As another example, in various implementations, the systems can be configured to be placed in air ducts and/or air conditioning vents.

The sanitization and/or disinfection solutions described herein are configured to emit electromagnetic (EM) radiation that can penetrate through walls, ceilings, pillars, columns, and other mechanical/structural elements to sanitize and/or disinfect. Accordingly, the systems and methods described herein do not require a direct line-of-sight to operate effectively. Furthermore, the systems and methods described herein can be configured to sanitize/disinfect over a wide range of distances, such as, for example, a few centimeters to tens of meters from the source of the EM radiation are not limited in the range over which they can operate effectively.

Various implementations described herein are directed towards a RF transmitter system configured to generate and/or emit EM radiation that can provide sanitization, therapeutic, anti-viral and/or anti-bacterial solutions. The RF systems and methods described herein are configured to utilize electromagnetic pulses to sanitize/disinfect and/or deactivate harmful organic materials, such as harmful biological material or organic pollution materials (e.g. carbon dioxide). Various implementations of the RF systems and methods described herein are configured to dynamically adjust power, wavelength, frequency, duty cycle and/or other parameters of the emitted radiation in the volume.

The systems, methods, modules, and devices of this disclosure each have several innovative aspects, no single one of which is solely responsible for the desirable attributes disclosed herein. A variety of example systems, modules, embodiments, and methods are provided herein below.

Various implementations of the RF system (also referred to herein as RF transmitter system) can be configured to generate and/or emit EM radiation at a plurality of frequencies that resonate with the frequency of one or more proteins, structures, or the ribonucleic acid (RNA) of the virus and destroys and/or deactivates the structures or RNA of the virus and/or harmful material.

Various implementations of the RF system comprise a source of EM radiation (e.g., an RF signal generator) that emits EM radiation (e.g., an RF signal), at least one RF amplifier to amplify the emitted radiation, devices and components to condition the amplified radiation and a control system configured to synchronize the turning on/off of the at least one RF amplifier with the presence/absence of the EM radiation. The control system is configured to dynamically adjust the bias voltages/currents provided to the at least one RF amplifier to increase/optimize efficiency, linearity and/or noise figure of the amplifier. The RF device/system can be configured to emit EM radiation at frequencies and power levels that are safe/harmless to the human body while being able to destroy, mitigate and/or neutralize a targeted harmful material (e.g., virus, bacteria, fungi, parasites, microbes, biological cells, proteins, and molecules).

Various embodiments of the RF system disclosed herein can be configured to generate and/or emit EM radiation that can sanitize a room, such as hospital rooms or manufacturing warehouses or malls or commercial or private places or office space or stadiums or schools etc. in a timescale greater than or equal to 1 millisecond and less than or equal to 30 minutes.

Various embodiments of the RF system can be configured to generate and/or emit EM radiation that can sanitize surgical instruments, surfaces, or objects in general. In some implementations the RF system can be configured to generate and/or emit EM radiation in a frequency range and/or at a power level that can safely be used for in-vivo applications. An example of such application may be wound disinfection.

Various embodiments of the RF system can comprise a receiver system configured to detect EM radiation scattered by or reflected from a target material. The receiver system can comprise a RF detector, processing electronics and an electronic memory. In various implementations, the receiver system can be a spectroscopic instrument. The EM radiation scattered by or reflected from the target material is provided as an input to the receiver system which electronically processes the received EM radiation to generate an absorption spectrum of the target material. By performing a comparison of the generated absorption spectrum with a set of data stored in the electronic memory, the processing electronics, identifies a type of target material (e.g., virus, bacteria, fungi, parasites, microbes, biological cells, proteins, RNA, and molecules) and/or one or more properties of the target material (e.g., physical property, a chemical property, or a surface property). In some implementations, the sensor system can use software, Artificial Intelligence (AI), and/or a Machine Learning (ML) program to identify the type and/or a property of the target material. The frequency and/or the power level of the emitted radiation can be changed based on the identification of the type and/or a property of the target material. In various implementations, a controller associated with the RF generator can be configured to dynamically vary the frequency or the power output of the EM radiation according to one or more properties of the target material.

Various embodiments of the RF system include a receiver system configured to detect EM radiation transmitted through the target material or scattered by the target material. An electronic processing system receives information corresponding to one or more absorption or scattered spectra and dynamically adjusts one or more parameters of the EM radiation based on the information received. For example, the output power or frequency of the EM radiation is dynamically adjusted based on the radiation absorbed and/or scattered by the target material. As another example, the control system is configured to adjust a bias voltage or current to a terminal (e.g., gate terminal, drain terminal) of an amplifying system of the RF system to adjust the output power of the EM radiation based on one or more properties of the target material.

Various embodiments of the RF system are configured to generate and/or emit EM radiation in a frequency range and/or at a power level that can inhibit or neutralize the growth of cancerous cells. The frequency range may be selected based on a resonant frequency of a structure, chemical, or material of the cancerous cells.

Various embodiments of the RF system are configured to generate and/or emit EM radiation in one or more frequency ranges resonating close to the resonant frequency of one or more proteins, structures, and/or RNAs of the virus or harmful material. This is effective to damage or destroy the harmful biological structures of bacteria, harmful material, viruses, or virus like particles (VLPs) present in an environment. In some implementations, the RF system may include a portable scanner that can detect the presence of bacteria, harmful material, virus and/or virus like particles (VLPs) in the environment, on a surface, or in a human body. As a result of detection of the presence of such material, the RF system can emit EM radiation at or around (e.g., ±5%) a resonant frequency of bacteria, harmful biological material, virus and/or VLPs to damage, destroy, deactivate, or otherwise neutralize them.

In various embodiments, the RF system comprises one or more sensors configured to detect the presence of humans in an environment. The RF system can dynamically adjust the frequency and/or the power level of the emitted radiation to comply with the human safety limits (e.g., power levels and/or frequencies that are in accordance with the federal communication commission (FCC) guidelines) upon detection of a human presence. Such systems may be useful to sanitize or sterilize the environment, object, or surface present in the environment and can destroy active bacteria, harmful biological material, viruses and/or VLPs.

The one or more sensors may include one or more types of sensors, such as a motion sensor, a temperature sensor, a proximity sensor, or an IR camera device to detect the presence of human, pets, computer equipment and/or other objects in an environment to be sanitized or sterilized.

The RF system can be configured as a compact device or a handheld device. In some implementations, the RF system is configured to be mounted on walls, ceilings, or doorways. In some implementations, the RF system is part of another system (for example, an air venting system).

In some implementations, the RF system can have a form factor that is capable of radiating in a large space. For example, the RF system can be configured to generate an RF beam with a beam width that is greater than or equal to about 10 degrees and less than or equal to about 150 degrees. In some implementations, the RF system can be configured to spatially change the beam direction using electro-mechanical techniques, digital beam forming techniques or a combination of both. In some implementations, the RF system can comprise a large antenna or an array of antennas. Such systems can be useful in sanitizing a large space such as, for example, airports, offices, stadiums, concert halls, schools, cruise ships, shopping complexes, hospitals, restaurants, naval ships, labs, airplanes, trains, etc.

Various implementations of the RF system configured to generate and/or emit EM radiation may employ one or more high power density circuits using Gallium Nitride (GaN) transistors that enable the electromagnetic pulse effect.

The RF system is, in some implementations, configured to use one or more high power density circuits using Gallium Nitride (GaN) amplifiers that can be controlled by one or more electronic processing systems. In some implementations, the frequency and/or the power level of the emitted electromagnetic radiation can be dynamically varied based on a type or characteristic of a target or the presence of humans in the vicinity of the system. In some implementations, the RF system may include a GaN field effect transistor (FET) amplifier and the control system thereof may be configured to adjust the bias voltage at a terminal (e.g., gate terminal, drain terminal) of the GaN FET amplifier to activate and/or deactivate the GaN FET amplifier. Such feature may advantageously improve the power efficiency in connection with generating bursts of EM radiation. By turning on the GaN amplifier when the radiation is emitted and turning off the GaN amplifier when no radiation is emitted, the RF system may be configured to emit bursts of high power EM radiation without requiring expensive cooling solutions. The circuit assemblies can allow the system to generate very short bursts of high power EM radiation that are within the safety guidelines prescribed by FCC.

The RF system may include solid state devices, transistors, amplifiers, diodes, and/or other tunable circuits to generate electronically and/or manually controlled RF radiation. In some implementations, the RF system includes programmable circuits configured to generate tuned waveforms that match or that are near (e.g., ±5%) the resonant frequency of the target material present in an environment. As a result of identifying the resonant frequency of the target material, the RF system may emit radiation to deactivate and/or neutralize the target material and/or to sanitize the environment.

Various implementations of the RF system disclosed herein may be configured to generate and/or emit EM radiation and include an electronic processing system and memory connected to the electronic processing system. The memory stores instructions that, when executed by the electronic processing system, causes the system to collect information from one or more sensors associated with the RF system. One or more control systems associated with the RF system can be configured to use the information from the one or more sensors to control and/or improve the performance of the GaN amplifier. The RF system may be configured to vary the frequency of the EM radiation emitted from the RF system. In some implementations, the RF system can vary the frequency of the emitted EM radiation to match or be near (e.g., ±5%) the resonant frequency of a component of a bacterium, a virus, fungi, parasites, microbes, biological cells, proteins, RNA, molecules, or some other harmful biological material present in an environment (or on a surface). The generated and/or emitted radiation can destroy/deactivate one or more viruses and/or VLPs, bacteria, and/or harmful biological material. In this manner, the RF system can be used to sanitize and/or disinfect objects/surfaces in an environment.

Various implementations of systems and methods disclosed in this application include a non-transitory computer readable storage medium having instructions stored thereon that, as a result of execution by one or more processing electronics, cause a RF transmitter to generate EM radiation. The RF transmitter can comprise an amplifying system having a gate and drain terminal. The generated EM radiation is directed towards a target material. The frequency and/or power output of the EM radiation dynamically adjusted to based on one or more properties of the target material. In various implementations, a bias power control system adjusts a bias voltage or current at a terminal (e.g., gate terminal, drain terminal) of the amplifying system to achieve a threshold range of one or more operational characteristics of the amplifier.

In various implementations the RF system can generate EM radiation in one or more frequency ranges, such as frequency ranges within the “UV band” and/or “UHV band” and/or “L-Band” and/or “S-Band” and/or C-band and/or X-band and/or Ku-band and/or “K-Band” and/or Ka-band.

In some implementations, RF system can generate and/or emit EM radiation at a frequency that can destroy and/or deactivate any further growth of virus, bacteria, fungi, parasites, microbes, biological cells, proteins, RNA, molecules and/or harmful biological material, without any need of intermittent scanning by the RF device in the environment.

In various other implementations, the RF system is configured to intermittently emit radiation sanitize/disinfect objects and/or surfaces in an environment. The intermittent emission of radiation can be performed at intervals of less than or equal to about 5 minutes, between about 5 minutes and about 10 minutes, between about 10 minutes and about 20 minutes, between about 20 minutes and about 30 minutes, between about 30 minutes and about 60 minutes, or any interval in a range within the above-identified numbers.

The systems, methods, modules, and devices of this disclosure each have several innovative aspects, no single one of which is solely responsible for the desirable attributes disclosed herein. A variety of example systems, modules, and methods are provided below.

In some implementations a system can include a transmitter configured to generate electromagnetic (EM) radiation characterized by a frequency and an output power and direct the generated EM radiation towards a target material in a volume, the amplifier having a gate terminal and a drain terminal, wherein the frequency or the output power of the generated EM radiation may be selected based on a type or a property of the target material, and a control system configured to adjust a bias voltage or a bias current provided to the gate terminal or the drain terminal of the amplifier based on the frequency or the output power. The target material can be selected from a group including viruses, bacteria, fungi, parasites, microbes, biological cells, proteins, RNA, and molecules. The frequency of the generated EM radiation can be resonant with a constituent of the target material. Further, the property of the target material can include at least one property of a physical property, a chemical property, or a surface property.

The system can further include a receiver system configured to receive EM radiation transmitted through the target material or scattered by the target material. The output power or the frequency of the EM radiation can be dynamically adjusted based on a parameter of the received EM radiation transmitted through the material or scattered by the material. The receiver system can include a radio frequency (RF) detector, a processing electronics, and an electronic memory. The receiver system can be configured to compare the received EM radiation with a set of data stored in the electronic memory, identify, based on the comparison, a type of the target material or a property of the target material, and provide information regarding frequency or output power to the transmitter based on the identified type of the target material or the property of the target material. The receiver system can further include a sensor. The receiver system can further include a spectrometer. Further, the frequency of the generated EM radiation can be safe for in-vivo applications. The transmitter can further include a sensor system configured to detect a presence of a human or an object in an environment surrounding the system. The frequency or the output power of the generated EM radiation can be dynamically adjusted based on an output of the sensor system. The sensor system can include at least one sensor of a motion sensor, a temperature sensor, a proximity sensor, or an infrared (IR) camera device. The system can further include a mounting plate configured to mount the system to a surface. The system can be configured to be handheld. The frequency of the generated EM radiation can be greater than or equal to about 200 MHz and less than or equal to about 100 GHz. Further, the generated EM radiation can be a pulsed waveform including a plurality of pulses having a pulse width and a duty cycle. The pulse width and the duty cycle may be variable.

A method can include generating electromagnetic (EM) radiation characterized by a frequency and an output power, via a transmitter that includes an amplifier having a gate terminal and a drain terminal, directing the EM radiation toward a target material disposed in a volume, dynamically varying the frequency or the output power of the EM radiation generated based on one or more properties of the target material, and adjusting a bias voltage or a bias current provided to the gate terminal or the drain terminal based on the frequency or the output power. The method can further include receiving, via a receiver system, radiation transmitted through or scattered by the target material, and controlling one or more characteristics of the EM radiation directed towards the target material based on a characteristic of the received radiation. The method can further include determining a resonant frequency of the target material based on the received radiation. Additionally, the method can further include adjusting a power level of the EM radiation over time to maintain a temperature of the target material below a threshold temperature. The method can also include determining absorption spectra of a plurality of target materials, and dynamically varying the frequency or the power output of the directed EM radiation based on the determined absorption spectra.

A non-transitory computer-readable storage medium having instructions stored thereon that, as a result of execution by one or more processing electronics, can cause a control system to generate electromagnetic (EM) radiation characterized by a frequency and an output power via a transmitter that includes an amplifier having a gate terminal and a drain terminal, direct the EM radiation toward a target material disposed in a volume, dynamically vary the frequency or the output power of the EM radiation generated based on one or more properties of the target material, and adjust a bias voltage or a bias current provided to the gate terminal or the drain terminal based on the frequency or the output power. The non-transitory computer-readable storage medium can cause the system to further receive the EM radiation emitted by the transmitter, and control one or more characteristics of the EM radiation directed towards the target material based on a characteristic of the received EM radiation. The non-transitory computer-readable storage medium can cause the system to further determine a resonant frequency of the target material based on the received EM radiation. The non-transitory computer-readable storage medium can cause the system to further adjust a power level of the EM radiation over time to maintain a temperature of the target material below a threshold temperature. The non-transitory computer-readable storage medium can cause the system to further determine absorption spectra of a plurality of target materials, and dynamically vary the frequency or the power output of the directed EM radiation based on the determined absorption spectra.

### BRIEF DESCRIPTION OF DRAWINGS

The disclosure is more fully appreciated in connection with the following detailed description taken in conjunction with the accompanying drawings, in which:

FIG. 1A is a simplified block diagram illustrating an RF system,

FIG. 1B is a simplified block diagram illustrating an RF system implemented with an antenna,

FIG. 2A is a simplified block diagram illustrating RF system, in accordance with certain aspects of the present disclosure,

FIG. 2B is a simplified block diagram illustrating RF system, in accordance with certain aspects of the present disclosure,

FIG. 3 illustrates another implementation of RF system directing RF radiations at a target material, in accordance with certain aspects of the present disclosure,

FIG. 4 illustrates a graphical representation of an absorption spectrum of the target material, in accordance with certain aspects of the present disclosure,

FIG. 5A illustrates another implementation of the RF system, in accordance with certain aspects of the present disclosure,

FIG. 5B illustrates an implementation of a control system, in accordance with certain aspects of the present disclosure,

FIG. 5C is a schematic illustration of an amplifier in the amplifier chain of the RF system that is being controlled by the control system, in accordance with certain aspects of the present disclosure,

FIG. 6 illustrates a flow chart of operations performed by the RF system, in accordance with certain aspects of the present disclosure,

FIG. 7A illustrates a flow chart of operations performed by RF system, in accordance with certain aspects of the present disclosure,

FIG. 7B illustrates another flow chart of operations performed by RF system, in accordance with certain aspects of the present disclosure,

FIG. 8 illustrates a system for training an artificial neural network, in accordance with certain aspects of the present disclosure,

FIG. 9 illustrates a simplified block diagram of an example computer system, in accordance with certain aspects of the present disclosure.

### DETAILED DESCRIPTION

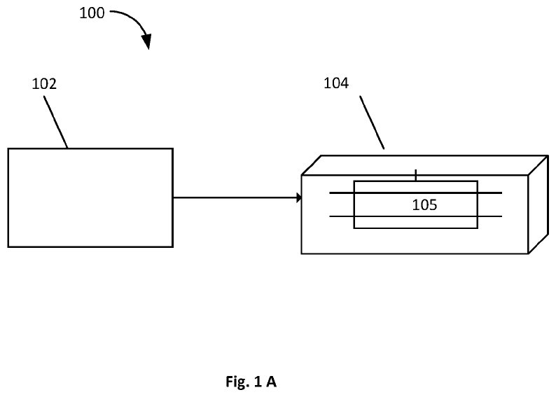

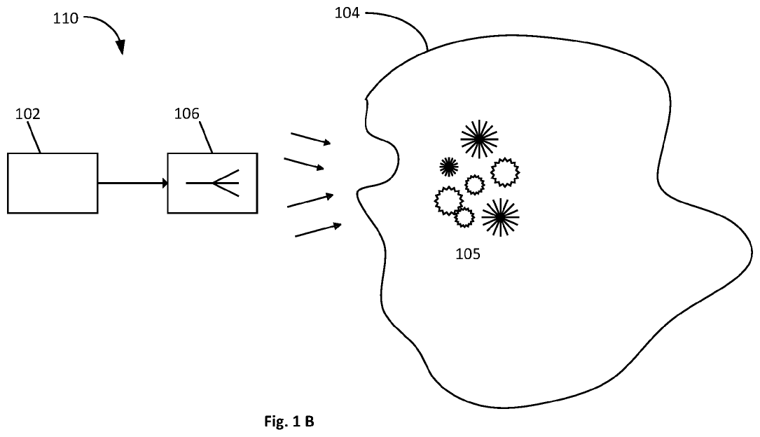

FIG. 1A illustrates an implementation of a radio frequency (RF) system 100 (or RF device). As shown in FIG. 1A, the RF system 100 comprises a RF transmitter that generates and transmits RF energy to a volume 104. The RF transmitter 102 can also be referred to as an RF transmit chain. The RF transmitter 102 can comprise digital RF synthesizers, one or more RF amplifiers, hybrid couplers, switches and other RF elements that form a part of the RF transmit chain. The volume 104 can be an enclosed volume. In some implementations, the volume 104 is a traverse electromagnetic (TEM) cell. The RF system 100 illustrated in FIG. 1A can be used to measure the effectiveness of various RF waveforms against a target material 105 present in the volume 104.

The target material 105, in some embodiments, is selected from a group of biological materials selected from viruses, bacteria, fungi, parasites, microbes, biological cells, proteins, RNAs, and molecules, and/or harmful organic material (e.g. carbon dioxide, methane, radon). The RF transmitter 102 comprises control circuitry configured to dynamically vary the frequency or the output power of the EM radiation based on one or more properties of the target material 105. For example, the wavelength, frequency, power level, duty cycle and other characteristics of the EM radiation emitted from the RF transmitter 102 can be adjusted manually or automatically to destroy/neutralize one or more properties of the target material 105. The one or more properties of the target material 105 comprises at least one property of a physical property (e.g., a structure), a chemical property, or a surface property.

FIG. 1B illustrates another implementation of a RF system 110 comprising the RF transmitter 102 and an antenna system 106. The antenna system 106 can comprise a single antenna or an array of antennas. The antenna system 106 is configured to radiate the EM radiation generated by the RF transmitter 102 toward the target material 105 present in the volume 104. In various implementations, the antenna system 106 can comprise a loop antenna, a horn antenna, a dipole antenna, a helical antenna, a dish antenna, a parabolic antenna, a monopole antenna, a rod antenna, an electronically steerable antenna array or other types of antennae.

The RF transmitter 102 can be configured to transmit energy in different frequency bands, for example, in a low frequency range (3-300 KHz), a high frequency range (300 KHz-30 MHz), a very high frequency range (30-300 MHz), an ultra-high frequency range (300 MHz-3 GHz), a super high frequency range (3-40 GHz), or a mega high frequency range (40-100 GHz). The frequency of the EM radiation emitted from the RF transmitter 102 can be in one or more of frequency bands, including the SHF band, the UHF band, the L band, the S band, the Ka band and/or the Ku band. The size and shape of the antenna system 106 can vary depending on a variety of factors including but not limited to the desired radiated power and the desired range. For example, if emission of EM radiation is used for deactivating and/or neutralizing harmful biological material in a small environment, a small sized antenna having an area between 4 square centimeter (cm) and about 60 square cm can be used. As another example, if emission of EM radiation is used for deactivating and/or neutralizing harmful biological material in a large environment (e.g., an open area), a large sized antenna and/or an array of antennae can be used. The size of large size antenna and/or an array of antennae can vary between 60 square cm to about 2 square meters.

The power of the radiation emitted from the RF transmitter 102 can be greater than or equal to about 1 W, less than or equal to about a few Megawatts, or less than or equal to a few Gigawatts. For example, the power of the EM radiation emitted from the RF system 100 can be less than or equal to about 10 W, between about 10 W and about 100 W, between about 100 W and about 500 W, between about 500 W and about 1000 W, between about 1000 W and about 3000 W, between about 3000 W and about 5000 W, between about 5000 W and about 10,000 W, between about 10,000 W and about 100 Megawatts, between about 1 Gigawatt and about 1 Gigawatt, greater than or equal to about 100 Gigawatts, or any value in a range defined by any of the above-identified numbers.

In various implementations, the electric field (E-field) generated by the RF system 110 can be in the range between about 10 volt per meter (V/m) and about 1000 Kilovolt per meter (kV/m). For example, in some implementations, the E-field generated by RF system 110 can be less than or equal to about 10 V/m, between about 10 V/m and about 100 V/m, between about 100 V/m and about 500 V/m, between about 500 V/m and about 1000 V/m, between about 1000 V/m and about 300 kV/m, between about 300 kV/m and about 500 kV/m, between about 500 kV/m and about 1000 kV/m, or any value in a range defined by any of the above-identified numbers.

In various implementations, the RF transmitter 102 can be configured to emit electromagnetic pulses (EMPs) towards the target material 105. When configured as a device configured to emit EMPs, the RF transmitter 102 can emit short bursts of high-power EM radiation to determine the absorption spectrum of the target material 105. The high-power EM radiation may be capable of creating a high voltage difference across the capsid of a virus, bacteria, fungi, parasites, microbes, biological cells, proteins, RNA, and molecules, sufficient to damage and/or destroy the capsid or other membrane of viruses, bacteria, fungi, parasites, microbes, biological cells, proteins, RNA, and/or molecules. In some embodiments, the strong E-field generated by the high-power EM radiation can polarize one or more viruses, proteins, bacteria, fungi, parasites, microbes, biological cells, molecules, and/or ribonucleic acid (RNAs) creating a dipole moment or affecting the permittivity thereof. The resulting strain from the polarization can lead to the destruction or neutralization of the one or more viruses, proteins, bacteria, fungi, parasites, microbes, biological cells, and/or RNA.

An advantage of configuring the RF transmitter 102 as an EMP system is that high peak powers can be achieved in a short interval of time while keeping the average power level of the RF system 110 low. This can be useful in reducing the overall power consumption of the RF system 110 and/or in meeting certain safety regulations. Without subscribing to any particular theory, the high-power EM radiation may be effective in deactivating and/or destroying the virus if the frequency of the EM radiation is substantially close (e.g., within ±5%) of a resonant frequency of one or more viruses, proteins, bacteria, fungi, parasites, microbes, biological cells, or RNAs of the target material 105. Accordingly, in some implementations of the RF transmitter 102, the frequency or output power level of the generated EM can be dynamically adjusted according to one or more properties of the target material 105, wherein the one or more properties of the target material comprise at least one property of a physical property, a chemical property, or a surface property.

The RF system 100 and 110 can be employed to sanitize an environment (e.g., hospital, office, home, retail places, hotels, etc.), appliances (e.g., surgical instruments, patient bed), and/or surface of various objects present in an environment or in a volume. As discussed above, one or more characteristics of the emitted EM radiation, such as, for example, wavelength, frequency, power level, etc., may be selected automatically by an electronic processing system or control circuitry, or selected manually by a user to destroy and/or deactivate one or more target materials 105 (e.g., virus, bacteria, fungi, parasites, microbes, biological cells, proteins, RNA, and harmful organic molecules) present in the environment, on the appliances, or a surface thereof.

In various implementations, the EM radiation emitted by RF system 100 and 110 may have characteristics selected to be safe for exposure to humans or animals while also deactivating and/or destroying viruses, bacteria, fungi, parasites, biological cells, proteins and/or other biological material that can be harmful to humans or animals. For example, a power level of the RF transmitter 102 can be dynamically adjusted to be within the safety limits prescribed by FCC while still being capable of destroying harmful viruses, fungi, parasites, microbes, biological cells, proteins, RNA, molecules, and/or other harmful biological material.

In various implementations, the RF transmitter 102 can be configured to dynamically adjust a frequency, a duty cycle, a wavelength, a power level, a pulse repetition frequency, and/or a pulse width of the emitted EM radiation based on one or more properties of the target material 105. For example, upon receiving an instruction from a user or a computer system, the frequency of the EM radiation emitted from the RF transmitter 102 can be tuned or dynamically adjusted by the control circuitry to a frequency or the output power level that matches or is near (e.g., ±5%) the one or more properties of the target material. The one or more properties of the target material may include and is not limited to a physical property, a chemical property, or a surface property.

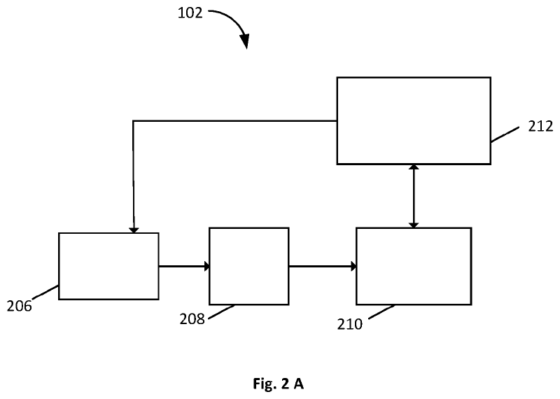

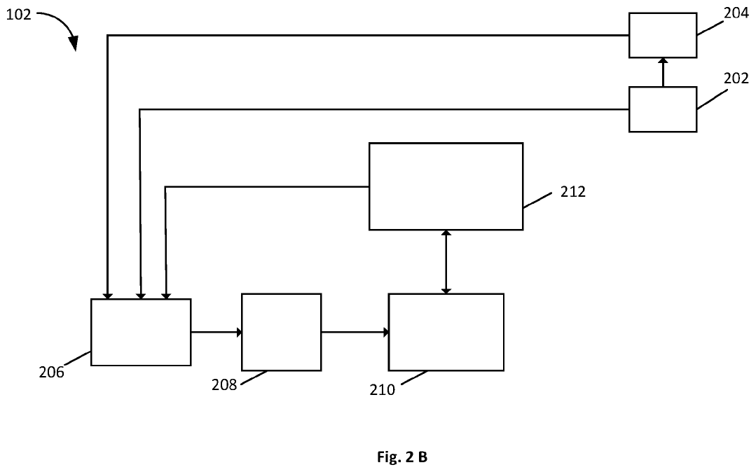

FIG. 2A schematically illustrates an implementation of the RF transmitter 102 according to one or more embodiments. The RF transmitter 102 comprises an electronic processing system 206, a source of RF signal generator 208, an amplifying system 210 configured to amplify the signal from the RF signal generator 208, and a control system 212. The electronic processing system 206 includes a computer or central processing unit, a data storage system, input devices, output devices, one or more network interfaces, and/or memory that stores instructions to be executed by one or more electronic processing system or processing electronics. The stored instructions may include data associated with classification of a target material—for example, data regarding target materials and/or one or more properties associated with the target materials. Examples of one or more properties of the target material comprises at least one property of a physical property, a chemical property, or a surface property. The electronic processing system 206 is configured to control the RF signal generator 208 that outputs the RF signals fed to the amplifying system 210. The amplifying system 210 is controlled and monitored by the control system 212.

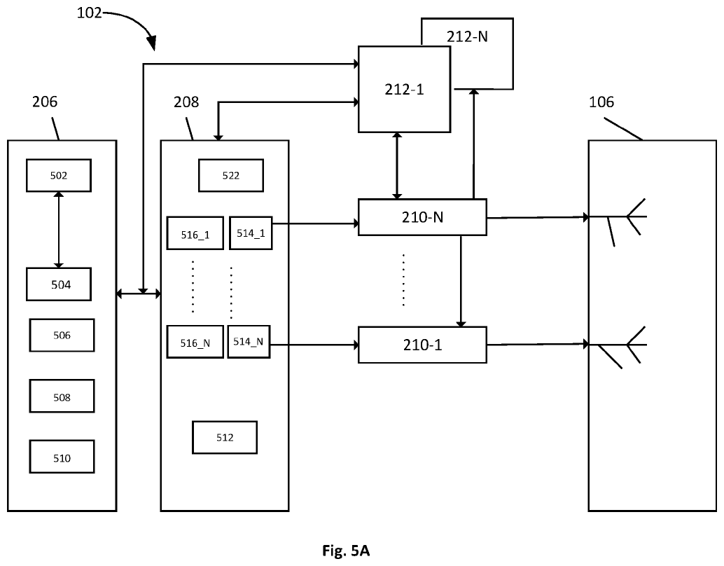

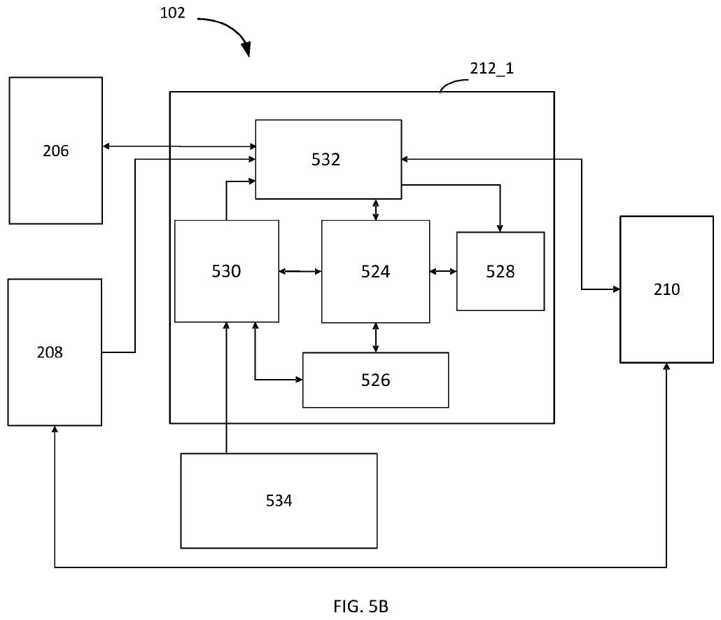

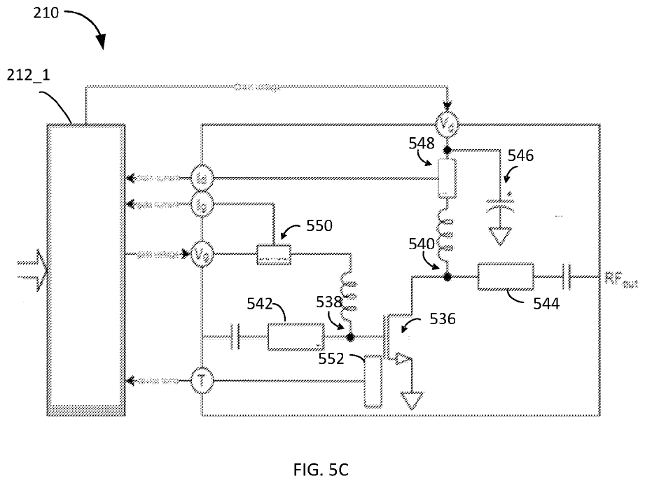

The control system 212 is configured to perform a variety of functions including but not limited to synchronizing the turning on/off of the amplifiers of the amplifying system 210 with the presence/absence of the RF signal and adjusting the bias voltages/currents of the amplifiers of the amplifying system 210 to improve performance (e.g., efficiency, output power level) of the amplifier(s) thereof. The control system 212 includes, in some embodiments, one or more sensor units 214 that detect conditions associated with the amplifying system 210, such as voltage at nodes within the amplifying system 210, current flowing between components in the amplifying system 210, and/or temperature of components within the amplifying system 210. Additional details of RF signal generator (also referred herein as, source) 208, amplifying system 210, including adjustment of the bias voltages/currents of the amplifiers of the amplifying system 210, is explained with respect to FIGS. 5A-5C.

The electronic processing system 206 may implement instructions, logic, software, one or more programs, or AI to collect and process data. The electronic processing system 206 may be configured to use the data to determine the target material and/or one or more properties of the target material. For example, the electronic processing system 206 may determine whether the target material includes viruses, bacteria, fungi, parasites, microbes, biological cells, proteins, RNA, or harmful organic molecules (e.g., methane, carbon dioxide, radon). Likewise, once the target material is identified, the electronic processing system 206 may determine one or more properties of the target material—for example, a physical property, a chemical property, or a surface property.

Based on the determined target material and/or one or more properties of the target material, electronic processing system 206 determines and provides one or more parameters of the RF signal to the RF signal generator 208. For example, the signal parameters may include frequency, duty cycle, power levels, pulse repetition frequency, pulse width, phase, angle of emission, position of the target material, timings of emission, height/depth of peaks/nulls in RF beam, etc. As another non-limiting example, based on an input received, the electronic processing system 206 can determine the types of target material, one or more properties of the target material, and dynamically adjust the frequency or output power of the EM radiation to achieve a threshold range for one or more operational characteristics. In various implementations, the electronic processing system 206 can be configured to adjust the frequency or the output power of the emitted EM radiation to be within the safety limits based on information regarding presence of humans in the vicinity. The electronic processing system 206 provides inputs regarding the determined characteristics of the EM radiation to be generated to the RF signal generator 208.

In various embodiments, the RF signal generator 208 can be implemented as an RF system on chip (RFSoC). The RFSoC may include a digital synthesizer that creates waveforms having various frequencies, pulse widths, pulse repetition intervals, and intra-pulse modulations specified by the RF waveform parameters generated by electronic processing system 206. In some implementations, RF signal generator 208 can also comprise digital to analog convertors (DACs), signal conditioning units (SCUs), frequency synthesizers, filters, gates, phase shifters, and/or multiplexers, by way of non-limiting example.

The signal generated by RF signal generator 208 is fed to the amplifying system 210. Amplifying system 210 may include one or more amplifiers or one or more chains of amplifiers. The volume 104 or the antenna system 106 is connected to output(s) of the amplifiers in some embodiments. The amplifiers of the amplifying system 210 can comprise a plurality of solid-state power amplifiers, such as, for example, silicon laterally diffused metal-oxide semiconductors, Gallium Nitride, Scandium Aluminum Nitride, Gallium Arsenide, and/or Indium Phosphide. In some implementations, one or more of the amplifiers of the amplifying system 210 can have a gate voltage on set point derived from an automatic calibration operation performed by the control system 212. The amplifying system 210 produces an amplified RF signal that is transmitted to the volume 104 or the antenna system 106. In some implementations, an RF signal from RF generator having a strength on the order of a few mW may be amplified to a power level between a few kWs and a few GWs.

In some implementations, the amplifying system 210 may include among other components, such as one or more high-power density circuits, one or more transistors, one or more amplifiers, one or more amplifiers modules, one or more biasing modules, one or more sensors, one or more control circuits, one or more combiner modules, or one or more transmission modules, by way of non-limiting example.

In some implementations, amplifying system 210 can have a wide frequency bandwidth capable of amplifying signals having frequencies that match or are near the resonant frequency of a constituent of the target material. For example, the amplifying system can be configured to amplify a plurality of frequencies in the L-band, S-band, or the K-band.

The control system 212 can govern the signals output from the amplifying system 210. The control system 212 may include one or more sensors configured to sense an output of the amplifying system 210 and adjust a bias voltage/current at the gate terminal or drain terminal of one or more amplifiers in the amplifying system 210, adjust a power level of the input signal to the amplifiers of the amplifying system 210, adjust a power level of the output signal from the amplifiers of the amplifying system 210, and/or sense a temperature of the amplifiers of the amplifying system 210. The control system 212 can be configured to adjust a bias voltage or a bias current of an amplifier of the amplifying system 210 based on the sensed characteristics of the amplifying system. In some embodiments, control system 212 monitors and controls the output of amplifying system 210 prior to being input to the volume 104 or the antenna system 106. In some variations, a power distributor, power controller or control circuits in control system 212 are configured to distribute power to the amplifiers of the amplifying system 210. In some variations, control system 212 can send information regarding sensed conditions or control of amplifying system 210 to electronic processing system 206. Further details of RF signal generator 208, amplifying system 210, control system 212 and electronic processing system 206 are explained with respect to FIGS. 5A-5C, 8 and 9.

FIG. 2B schematically illustrates an implementation of RF transmitter 102 according to one or more embodiments. The RF transmitter 102 illustrated in FIG. 2B may include a camera module 204, a sensor module 202, the electronic processing system 206, the RF signal generator 208, the amplifying system 210, and/or the control system 212. Various features and details of electronic processing system 206, RF signal generator 208, amplifying system 210, and control system 212 may be configured as described with respect to FIG. 2A.

The camera module 204 is, in some embodiments, configured to capture images of the environment, images of surfaces therein, images of objects, images of human, images of target material and provide those images or image data to the electronic processing system 206.

The sensor module 202 may include one or more sensors or different types of sensors to sense target material and/or one or more properties of the target material. For example, one or more sensors may sense details regarding the type of environment (e.g., size of environment, shape of environment), a type of the target material (e.g., viruses, bacteria, fungi, parasites, microbes, biological cells, proteins, RNA, and harmful organic molecules) or details regarding objects present in the environment (e.g., physical property, chemical property, surface property), or presence and/or proximity of a humans or animals in an environment. The sensor module may feed the sensed data to the camera module 204 and/or the electronic processing system 206. Non-limiting examples of sensors in the sensor module 202 include motion sensors to senses the presence of living or moving object, vision sensors (e.g., infrared imaging sensors, ultraviolet imaging sensors), proximity sensors, motion sensors, position sensors, time-of-flight sensors, Lidar sensors, and/or electrochemical sensors. The sensor module 202 senses the objects, surfaces, or target materials, including related information (e.g., type of object, presence of human, type of surface, type of target material, physical property, a chemical property, or a surface property, etc.) regarding an environment.

In some implementations the camera module 204 may be configured to receive the sensor information from sensor module 202 and may combine the image information with the information (or data) received from the sensor module 202. The combined information may be provided to the electronic processing system 206.

The electronic processing system 206 is, in some embodiments, configured to rely on one or more Neural Network (NN) or AI models that implement data analysis and/or classification techniques to classify and generate various parameters based on the combined information received from camera module 204 and/or sensor module 202. The electronic processing system 206 processes the camera image data and/or sensor information to determine the target material and/or one or more properties of the target material. For example, the electronic processing system 206 may determine, using software or AI models, whether the target material includes viruses, bacteria, fungi, parasites, microbes, biological cells, proteins, RNA, and/or molecules of otherwise harmful material (e.g., pollution). The electronic processing system 206 also determines one or more properties of the target material, for example, a physical property, a chemical property, or a surface property. The electronic processing system 206 may, in connection with identifying or analyzing a target material and various properties thereof, for example, determine a type of an object in the environment (human, surface or type of object or size or shape of instrument), a distance and/or direction of object relative to the antenna system 106, or an action performed by a human or animal in the environment (e.g., sneezing, coughing, ambulation, motion).

The electronic processing system 206 is, in some embodiments, configured to determine the type of environment (indoor, outdoor etc.), details regarding the volume 104 (e.g., shape or size of volume), and/or the presence of harmful biological target material. Based on the information received from camera module 204 or sensor module 202, the electronic processing system 206 provides signal parameters related to waveform to RF signal generator 208.

In some implementations, the electronic processing system 206 may be coupled to memory storing target classification data, such as data indicating types of target material and the associated properties of the target materials. The memory may store instructions that, when executed by the electronic processing system 206, cause the electronic processing system 206 to classify a target material and various attributes associated with the target material based on data accessed in the memory. In some implementations, the electronic processing system 206 can refer one or more look up tables (LUTs) to generate various parameters associated with the power signals. In some implementations, the electronic processing system 206 is configured to select a waveform from among a plurality of waveforms stored in the memory. In some embodiments, the electronic processing system 206 is configured to select or generate RF waveform parameters according to a stored data structure, such as a waveform LUT, based on the target material or attributes thereof. In various other implementations, the electronic processing system 206 may obtain other stored information including but not limited to detected or desired operating conditions, pre-amplified power characteristics, and types or characteristics of pulses required to generate signals, by way of non-limiting example.

Based on the RF waveform parameters received from the electronic processing system 206, the RF signal generator 208 can generate RF signals. The generated RF signals are input to the amplifying system 210. The control system 212 is configured to adjust the bias voltages and currents provided to the one or more amplifiers of the amplifying system 210 as discussed above. In various implementations, control system 212 adjusts a level of bias voltage/current provided to the gate terminal or drain terminal of the amplifier based on the frequency or the output power of the generated RF signal. As discussed above, the RF system 100 or 110 can be tuned (automatically or manually) to generate EM radiation with a plurality of frequencies that can effectively neutralize/destroy/deactivate the target material.

In some implementations, RF system 100 or 110 may use one or more software programs and/or simulation results to determine one or more frequency range viable to deactivating the harmful biological material in the target material 105 and/or perform the sanitization or sterilizations.

In some implementations, RF system 100 or 110 can use one or more frequency ranges that comply with the FCC safety guidelines and that are harmless to the human cells while damaging the harmful biological materials.

In various implementations, the RF system 100 or 110 can be configured to output radiation having an electrical field strength in a range between about 10 V/m and about 1000 V/m.

The RF system 100 or 110 can be configured to have various sizes, weights, and/or form factors. In some embodiments, the RF system 100 or 110 is a portable device. In some embodiments, the RF system 100 or 110 may include a mounting plate configured for mounting the RF system to a surface. The emission of EM radiation can vary accordingly based on the configuration of RF system.

In some implementations, the maximum electric field strength of radiations emitted by the RF system 100 or 110 can vary based on a distance. For example, based on the distance between the RF system 100 or 110 and the object or target material present in an environment or based on the distance between the RF system and the surface. In some implementations, the emission of radiations by RF system 100 or 110 at a distance of 1 meter from a target material may generate an electric field strength ranging between about 10 V/m to about 1000 V/m.

In some implementations, the RF system 100 or 110 may have a handheld form factor or be configured to be otherwise handheld. By way of non-limiting example, the RF system 100 or 110 may have a size less than or equal to 1.5 cubic feet. In some implementations, the RF system 100 or 110 may include a handle and/or a grip, and may include one or more input devices for controlling operation of the RF system 100 or 110. In some implementations, the handheld RF system 100 or 110 may have a weight of 20 lbs. or less. In some implementations, the RF system 100 or 110 may include a first portion configured to be handheld and a second portion configured to be worn on another portion of the body, such as on the back (e.g., as a backpack) or around the waist.

In some implementations, to address the human safety concerns, the RF system 100 or 110 can be configured to emit radiation in a non-ionizing regime.

In some implementations, considering the safe emission of RF radiation to the human cells, the emission of radiations can map out to the “safe distance” from the most power version of the RF system 100 or 110. In some implementations, the RF system can adjust (or adopt) the emission of radiations based on the proximity of or distance to the object or surface or human or target biological material, etc., present in the environment or in a volume such as for example, the volume 104. In some implementations, the RF system 100 or 110 can operate at a safe distance as a function of angle measured from RF system 100 or 110 as according to long-term exposure limits from the FCC. In some implementations, the safe distance from RF radiation and/or from the RF system 100 or 110 is determined about 1 meter (m) to about 50 m.

In some implementations, the RF system 100 or 110 is configured to perform the sanitization operations almost instantly, in as little as about 5 nanoseconds. For example, 5 nanoseconds of these high levels RF radiation can be safe for human exposure.

In some implementations, to comply with the human safety requirements and to address the harmful biological materials and/or sanitization operations, RF system 100 or 110 is configured to dynamically adjust operating parameters (e.g., frequency of EM radiation, power level of EM radiation) to maintain the safe operation with respect to objects, surfaces, humans, animals, electronics, etc., in the environment.

In some implementations, the RF system 100 or 110 automatically measures the E-field strength at precise time scales including increments from 5 nanoseconds to 100 microseconds to compute and adjust the exact energy level at which the viruses, VLPs, bacteria, fungi, parasites, microbes, biological cells, proteins, RNA, molecules and/or harmful biological material can be deactivated or destroyed.

In some implementations, the RF system 100 or 110 is configured to execute one or more software programs or sets of instructions to adjust a frequency associated with emission of electromagnetic pulses (EMPs). The RF system 100 or 110 may be configured to execute one or more software programs or sets of instructions to modulate the frequencies according to a resonant frequency of e.g., viruses or VLPs and that are less effective to the particles with whom the frequency does not resonate. For example, according to Equations (1)-(4) below, and particularly Equation (3), it is advantageous to modulate the frequency via execution of software of the RF system 100 or 110. In this way, the frequency of EM radiation can be tuned to match or be near (±5%) the resonant frequency of a virus or a virus-like-particle and be about 30 dB (or 3 orders of magnitude) less effective against other particles having the same or similar resonant frequency. Various approaches that can further define the usage of various frequency ranges that can resonate with harmful biological materials and that are safe to human cells, as further defined as below.

Several approaches that are important in modelling electromagnetic pulse (EM) effects have been considered. However, one approach that has been used, has very high correlation to measured data, assumes energy gets rectified onto an electromagnetic equivalent of an “antenna” of a target. For example, received power on target, PR, is modeled as a function of the received power density, SR, incident onto a multi-frequency antenna, as defined in the following Equation (1):

$${P}_{R} ( λ , θ , φ ) = {S}_{R} \frac{{}^{\cdot } {λ}^{2}}{4 π} G ( θ , φ ) \cdot p ( θ , φ ) \cdot α ( λ ) \qquad ( 1 )$$

Various improvements relative to previous approaches are made by introducing a new coupling coefficient that is a function of wavelength, 0≤α(λ)≤1, and that the polarization loss is a multivariate term 0<p(θ, φ)<1 as a function of the angle of incidence onto the wire. In general, it was found that a wave in plane with the printed circuit board (pcb) or length with the wire results in the highest received power density, p(θ, φ)=1. In general, this function drops off as the cosine of the difference in angle from the plane of the wire or printed circuit board, as defined in the following Equation (2):

$$p ( θ , φ ) = cos ( θ ) cos ( φ ) \qquad ( 2 )$$

Next, the coupling coefficient, α(λ) is highly correlated with how closely the half-wavelength value of the transmitted wave, λ_{eff}/2 is matched to the effective length of the wire(s) or trace(s) on the target, as defined in the following Equation (3):

$$α ( λ ) = max {\{ {Dipole}_{i} ( λ ) \}}_{i = 1 … N} \qquad ( 3 )$$

The function α(λ) selects the maximum value of the Dipole function of all N “antenna” on the target, where the Dipole(x) function is the coupling coefficient onto a dipole antenna element represented by the i^{th} antenna on the target. Dipole(λ)=(1−k·s₁₁(λ)) where k is an empirical constant that equals 100 in our data and s₁₁(λ) is the value of the s₁₁ function of the i^{th} antenna at the frequency corresponding to wavelength λ. Resonance is achieved at L=n·λ_{eff/}2 where n is an integer and λ_{eff}/2=300/(2·f_{GHz}\sqrt{ε_{eff}}), where ε_{eff} is the effective dielectric constant of the antenna, Lis length of the trace, and f_{GHz} is the frequency of the incoming wave. The better matched the wavelength is to the effective length of the antenna on the target, the closer to unity α(λ) is and the stronger the coupling. This study focuses on confirming this model applies to biological material, including viruses and VLPs.

It is to be noted that a very slight change in frequency can have an enormous (orders of magnitude) effect on the coupling coefficient, α(λ). In order to improve energy incident on a target, a MHz frequency resolution across 100s MHz of bandwidth is required on modern targets, which can cause up to about 30 dB of power difference required for an EMP device “kill.” By closely matching the frequency of radiation emitted to a resonant frequency of a target material, the amount of power required to destroy or deactivate target material is significantly reduced relative to other mechanisms. Thus, methods and systems disclosed herein achieve significant power advantages.

The destruction of a virus or other target biological material is, in at least some situations, dependent on the unintentional voltage imposed on the target material, which affects the energy absorbed on the target material and not just the received power. In the following Equation (4), the energy incident is a function of the amount of received power onto the target material:

$${E}_{R} ( λ , θ , φ , τ ) = {\int }_{0}^{{τ}_{pulse}} P ( λ , θ , φ ) \cdot Δ ( λ ) d τ \qquad ( 4 )$$

More particularly, Equation (4) gives the function for integrating the received power onto the target material, over the pulse length time interval.

It is observed that there are two effects that can cause damage to the virus or biological material by electromagnetic pulse. One is an effect known as resonance. An electromagnetic field ‘couples’ to virus like particles at certain frequencies. These studies have shown that at a high enough electric field at the correct resonance, creates a high enough voltage potential across the capsid of the virus that it damages and damages or destroys the capsid, membrane, or other structure of the target material and thus destroys the viruses, bacteria, fungi, parasites, microbes, biological cells, proteins, RNA, and molecules. The RF system 100 or 110 collects the measured data from various sensors coupled thereto and determines the resonant frequency of SARS-COV2 virus, other virus, or harmful biological material in general using the HESS electromagnetic field simulator. The RF system 100 or 110 generates EM radiation having characteristics sufficient to match or be near the resonant frequency of one or more harmful biological materials.

In some embodiments, a second mechanism by which the biological or target materials are destroyed is the polarization of electromagnetically sensitive RNA strands. The second mechanism for virus destruction is a strong E-field that polarizes the structure or RNA strains (dipole moment) of the virus and destroys its structure. In some embodiments, a third mechanism by which the biological or target materials are destroyed is the dielectric breakdown of a cell wall, which is effective for deactivating and/or destructing the biological material. The RF system 100 or 110 can emit the radiations such that the lipid bilayer, protein coat, or other structure (e.g., peptidoglycan) of a virus or other biological material may experience a dielectric breakdown to cause rupture and/or break the cell wall and deactivate the viruses, bacteria, fungi, parasites, microbes, biological cells, proteins, RNA, or molecules. In some implementations, the E-field generated by the RF system 100 or 110 can be configured to dielectrically breakdown the lipid bilayer of biological or target materials. For example, the E-field can be in a range from about 1 MV/m to about 100 MV/m (e.g., 40 MV/m). To perform these mechanisms, the RF system 100 or 110 can generate EM radiation that is safe for humans and can destroy the active viruses or other biological material.

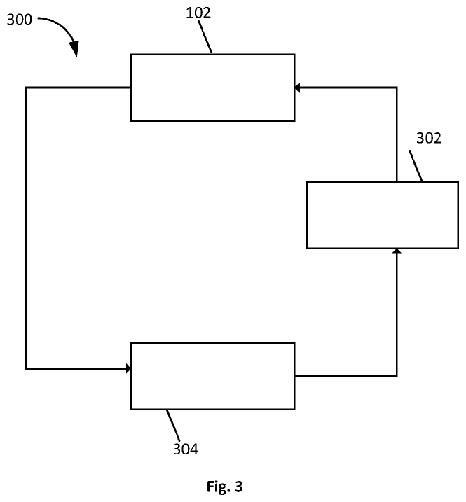

FIG. 3 illustrates another implementation of an RF system 300 configured to direct RF radiation at a target material in a volume 304. The RF system 300 illustrated in FIG. 3 comprises an RF transmitter 102, a receiver system 302 and a volume 304. The RF transmitter 102 is similar to as explained above in FIGS. 1A, 1B, 2A, 2B, and elsewhere herein. The receiver system 302 can comprise a RF detector, processing electronics and an electronic memory. In some implementations, the receiver system 302 may comprise a sensor system configured to sense EM radiation scattered by or reflected from the target material. In some implementations, the receiver system 302 can comprise an antenna system configured to receive the radiation scattered by or reflected from the target material. In some implementations, the receiver system 302 can be configured as a spectroscope/spectrometer or spectroscopic instrument. The volume 304 can be a biological material or target material present in a volume or in an environment or an enclosure (e.g., TEM cell).

The RF transmitter 102 sends out dynamically modified RF radiation to the target material in the volume 304. The receiver system 302 is placed in proximity to the target material. The receiver system 302 senses the amount of radiation that is absorbed by and/or reflected by the target material in the volume 304.

As explained above, RF system 100 is configured to generate dynamically modified EM radiation, and the generated EM radiation are directed towards a target material. The RF transmitter 102 includes a controlling circuit configured to dynamically vary the frequency or the output power of the EM radiation and adjusts a bias voltage or current of the amplifiers based on one or more properties of the target material in the volume 304. In other words, the RF transmitter 102 includes bias power controlling circuitry that adjusts the voltage or current provided to the gate terminal or drain terminal of the amplifying system, allowing for control over the output power of the EM radiation.

The target material (e.g., viruses, bacteria, fungi, parasites, microbes, biological cells, proteins, RNA, and molecules can partially absorb and partially reflect the radiation from the RF transmitter 102. For example, the RF radiation may be fully or partially absorbed by the target material based on one or more properties of the target material. The amount of radiation absorbed or reflected by the sample are sensed by the receiver system 302.

The receiver system 302 receives the amount of radiation absorbed by the sample (or target material) in the volume 304, and/or amount of radiations reflected by the sample in the volume 304. The processing electronics of the receiver system 302 receives signals or data indicative of the absorption spectrum or sensed radiation information from sample in the volume 304 and provides feedback to RF transmitter 102. The RF transmitter 102 receives the feedback input from receiver system 302 and uses the received information for real time adjustments to the frequency and power output of the EM radiation. The receiver system 302 electronically processes the received EM radiations to generate an absorption spectrum of the target material. The receiving system includes an electronic memory or database that stores a set of data that is used for comparison with the sensed radiation information. For example, by comparing the absorption spectrum with a set of data stored in the electronic memory, the processing electronics identifies a type of target material (e.g., virus, bacteria, fungi, parasites, microbes, biological cells, proteins, RNA, and molecules) and/or one or more properties of the target material (e.g., a physical property, a chemical property, or a surface property). In various implementations, the processing electronics of the receiving system can use software, artificial intelligence, and/or a machine learning program to identify the type and/or a property of the target material. Receiver system 302 sends the radiation spectrum information as feedback to RF transmitter 102.

RF transmitter 102, based on the received information from receiver system 302, generates EM radiation having a frequency and power level that is sufficient to affect one or more properties of the target material. The feedback information from receiver system 302 allows for real-time adjustments to the frequency and power output of the EM radiation to achieve the desired level of absorption or reflection by the target material. Various implementations of dynamically adjusting the frequency, power output of EM radiation and/or adjusting the bias of gate terminal or drain terminal of the amplifying system by control system are further explained with respect to FIGS. 5A-5C.

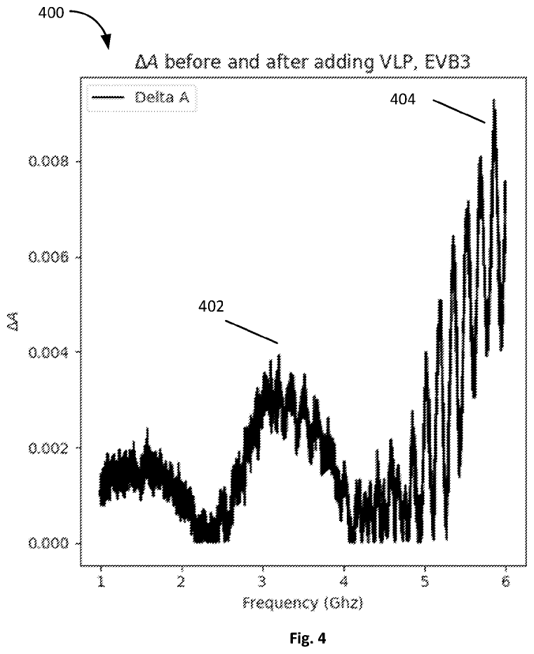

FIG. 4 illustrates a graphical representation 400 of an absorption spectrum of the target material based on the radiations absorbed by the target material.

As can be seen from the graphical representation 400, peak absorption frequencies for target material are observed in various ranges. For example, a first peak absorption frequency 402 (e.g., for virus, bacteria, fungi, parasites, microbes, biological cells, proteins, RNA) is observed at around 3.1 GHz and a second peak absorption frequency 404 is observed around 6 GHz. Additionally, it is observed that absorption of EM radiation at the second peak absorption frequency 404 by a target material (e.g., virus, bacteria, fungi, parasites, microbes, biological cells, proteins, RNA, and molecules) is higher than absorption of EM radiation at the first peak absorption frequency 402. As seen in FIG. 4, frequency dependent susceptibility is effective on one or more properties of the target material and the RF systems described herein can be utilized to neutralize these harmful biological target materials.

The graphical representation 400 provides a clear visualization of the absorption pattern of the virus, bacteria, fungi, parasites, microbes, biological cells, proteins, RNA, and molecules or like particles, which can be used to identify and distinguish them from other materials. By using the RF system described above, which includes a RF transmitter, a sample, and a receiver with a spectroscope, it is possible to detect the amount of radiation absorbed by the target material and using this information to dynamically adjust the frequency and power output of the EM radiation to effectively target, detect and destroy virus, bacteria, fungi, parasites, microbes, biological cells, proteins, RNA, and harmful organic molecules or particles.

FIGS. 5A-5C illustrates another implementation of RF system described above with reference to FIGS. 1A, 1B, 2A, 2B and 3. More particularly, FIGS. 5A-5C illustrate a control system to adjust bias conditions of RF amplifiers in amplifying system.

FIG. 5A illustrates an implementation of the RF transmitter 102 of FIGS. 1A, 1B, 2A and 2B. The RF transmitter 102 is illustrated as being augmented with a plurality of control systems 212_1 to 212_N configured to provide desired voltages and currents to efficiently operate the amplifiers in the amplifier chains 210_1 to 210_N. In some embodiments, the electronic processing system 206 is coupled to memory 502 storing instructions that, as a result of execution by the electronic processing system 206, cause the electronic processing system to perform various operations described herein. In some embodiments, the memory 502 may store target classifier instructions 504 that, when executed, cause the electronic processing system 206 to classify or identify a target material. e.g., based on the absorption spectrum of the target material. The memory 502 may store waveform selection instructions 508 that, when executed, cause the electronic processing system 206 to select a waveform from one or more waveform data structures 506 (e.g., LUTs) based on the environment (e.g., size of room, shape of room, objects in room), the type of target material present in the environment, the distance to the target material, or other factors. In some embodiments, the memory 502 stores power monitoring instructions 510 that, when executed, cause the electronic processing system 206 to monitor power consumption by or efficiency of the RF signal generator 208, the amplifying system 210, and/or the control system 212.

In various implementations, the RF signal generator 208 is implemented as an RF system on a Chip Field Programmable Gate Array (RFSoC FPGA). The RF signal generator 208 includes a gate array 512 and a direct digital synthesizer 522 that creates waveform of the frequency, pulse width, pulse repetition interval and intra-pulse modulation specified by the RF frequency waveform parameters generated by the electronic processing system 206. The gate array 512 is configured to perform a variety of functions including but not limited to determining the time intervals at which different components of the amplifier is powered up and powered down. The waveforms are passed to a collection of digital-to-analog converters (DACs) 516_1 through 516_N. Outputs from DACs 516_1 through 516_N are optionally conditioned by signal conditioning units (SCUs). In various implementations, the SCUs can comprise filters 514_1 through 514_N as depicted in FIG. 5A. The filters 514_1 through 514_N may filter the RF signals to a frequency band of interest, e.g., based on type of target material to addressed. The electronic processing system 206 together with the RF signal generator 208 can be configured to dynamically vary the frequency of the RF radiation that is output from the RF transmitter 102. The outputs from the RF signal generator 208 are applied to amplifier chains 210_1 through 210_N.

In various implementations, the individual control systems 212_1 to 212_N can comprise or be associated with a power distributing system and/or a power sequencing system that is configured to sequentially activate the amplifier chains 210_1 to 210_N. Individual control systems 212_1 to 212_N are configured to (i) in response to receiving a signal from the RF signal generator 208, provide appropriate bias voltages and currents to turn-on the amplifiers in the corresponding amplifier chains 210_1 to 210_N prior to and/or synchronously with the arrival of the RF signal from the RF signal generator 208; (ii) adjust or change the bias voltages and currents to the amplifiers of the amplifier chains based on information obtained about the input signal characteristics, output signal characteristics, system operating conditions (e.g., operating temperature, operating currents/voltages at various terminals of the amplifier/system, etc.), an input received from a user or an electronic processing system controlling the biasing systems and/or by information obtained from look-up tables that provide an understanding of the state of the amplifier; and/or (iii) reduce the bias voltages and currents to turn-off the amplifiers in the corresponding amplifier chains 210_1 to 210_N in response to absence of signal to be amplified or a sensed characteristic (e.g., input signal power, output signal power, temperature, gate current/voltage or drain current/voltage) being outside a range of values.

As discussed above, the plurality of control systems 212_1 to 212_N can comprise sensors (e.g., current sensors) that can sense current values (e.g., drain and/or gate current values) of the individual amplifiers in the amplifier chains 210_1 to 210_N. The control systems 212_1 to 212_N can be configured to sense the current values of the individual amplifiers in the amplifier chains 210_1 to 210_N intermittently (e.g., periodically). In some implementations, the control systems 212_1 to 212_N can be configured to sense the current values of the individual amplifiers in the amplifier chains 210_1 to 210_N continuously. In various implementations, the output from the current sensor can be sampled using an analog to digital converter (ADC) and averaged over a number of samples (e.g., 128 samples, 512 samples, etc.) to obtain the sensed current value.

The sensed current value can be analyzed by the control systems 212_1 to 212_N to determine an operational or a physical characteristic (e.g., temperature, input/output signal power, voltage/current at various terminals of the amplifier) of the individual amplifier. For example, a sensed current value above a first threshold current value when the amplifier is not turned on can be indicative of a defect in the amplifier or a defect in the circuit board on which the amplifier is mounted. As another example, a sensed current value above a second threshold current value when the amplifier is turned on but no signal to be amplified is provided to the input can be indicative of a defect in the amplifier or a rise in the temperature of the amplifier. As yet another example, a sensed current value above a third threshold current value when the amplifier is turned on and a signal to be amplified is provided to the input can be indicative of a defect in the amplifier or a rise in the temperature of the amplifier. Accordingly, the control systems 212_1 to 212_N can be configured to compare individual amplifier current values to target amplifier current values to identify an amplifier state error. In response to determining that the amplifier current value of a particular amplifier has deviated from a target amplifier current value (e.g., first, second or third threshold values discussed above), the control system controlling that particular amplifier is configured to determine the amount by which values of the voltages/current provided to the amplifier should be offset to achieve efficient operation of the amplifier and provide that offset value. As another example, the control system adjusts the current value to a threshold value to turn on and off the amplifiers of amplifier chains 210_1 to 210_N based on the one or more information of the target material or one or more property of the target material. In various implementations, one or more of tasks of correlating the sensed current values to a physical characteristic of the amplifier or determining the amount by which values of the voltages/current provided to the amplifier should be offset by to achieve efficient operation of the amplifier to address the target material can be performed by the electronic processing system 206 instead of the control systems 212_1 to 212_N.

The target amplifier current values may be based upon several factors for optimal system operation. For example, the target amplifier current values may be calibration amplifier current values for specified temperatures. The target amplifier current values may be calibration amplifier current values to compensate for amplifier manufacturing process variations. The target amplifier current values may be calibration amplifier current values to compensate for voltage variations. The target amplifier current values may be calibration amplifier current values to compensate for radio frequency phase variations. The target amplifier current values may be historical performance amplifier current values. The historical performance amplifier current values may be used to identify amplifier degradation over time.

Without any loss of generality, the plurality of control systems 212_1 to 212_N can comprise a variety of sensors. For example, the plurality of control systems 212_1 to 212_N can comprise voltage sensors configured to measure voltages at the various parts of the amplifiers in the amplifier chains 210_1 to 210_N. As another example, the plurality of control systems 212_1 to 212_N can comprise temperature sensors configured to measure temperature of the amplifiers in the amplifier chains 210_1 to 210_N. The temperature sensors can be configured to measure the device temperature of the amplifiers in the amplifier chains 210_1 to 210_N or temperature of the housing or the mount on which the amplifiers in the amplifier chains 210_1 to 210_N are disposed.

FIG. 5B illustrates an implementation of the control system 212_1. The control system 212_1 can include various functional sub-systems, such as an electronic processing system 524, a control system 526, a memory (not shown), a sensing system 528, a power adapting system 530, and an input/output system 532. The various functional sub-systems can be integrated in a single housing or in separate housings. In implementations where the different functional sub-systems are integrated in separate housings, the separate housings can include processing electronics and communication systems to communicate and function properly. For example, in some implementations, the power adapting system 530 and the sensing system 528 can be integrated in a separate housing. In such implementations, the electronic processing system 524 in cooperation with the control system 526 and the memory can provide signals to the power adapting system 530 to turn-on/turn-off the biasing voltages and currents to the amplifiers in response to receiving a signal from the RF signal generator 208 indicating the start/end of the RF signal and/or receiving information from the sensors that one or more sensed parameters are out of a range of values.

The control system 212_1 can be implemented with a form factor of a field programmable gate array (FPGA) or an application specific integrated circuit (ASIC). The ASIC implementation may be advantageous to realize smaller form factors. The control system 212_1 is configured to obtain information about the signals to be amplified and monitor various currents and voltages of the amplifier to optimize and control operating currents and voltages of the amplifier. The control system 212_1 can obtain the information about the signals to be amplified and the currents/voltages at various terminals of the amplifier in real time or substantially in real time. For example, the control system 212_1 can obtain the information about the signals to be amplified and the currents/voltages at various terminals of the amplifier in a time interval less than about 1 second, in a time interval greater than or equal to about 1 millisecond and less than about 1 second, in a time interval greater than or equal to about 1 second and less than about 10 seconds, in a time interval greater than or equal to about 10 seconds and less than about 30 seconds, in a time interval greater than or equal to about 30 seconds and less than about 1 minute and/or in a range defined by any of these values. As yet another example, control system 212-1 can obtain information about one or more property of the target material from CPU or one or more sensor and adjust the bias voltage or current at gate and/or drain terminal of the amplifier to generate the precise amount of signal.

The control system 212_1 can provide several benefits including but not limited to increasing/optimizing power efficiency for a desired performance criterion. For example, consider that an amplifier in the amplifier chain 210_1 being controlled by the control system 212_1 is operated in a high gain regime to provide a certain amount of RF output power for address the one or more property of the target material. The power efficiency of the amplifier can be higher than a similar amplifier that is operated in a high gain regime to provide the same amount of RF output power but is not controlled by the control system 212_1. As another example, consider that an amplifier in the amplifier chain 210_1 controlled by the control system 212_1 is operated to provide a certain amount of gain and linearity. The power efficiency of that amplifier can be higher than a similar amplifier that is operated to provide the same amount of gain and linearity but is not controlled by the control system 212_1. The use of the control system 212_1 can also reduce direct current (DC) power consumption during operation of an amplifier as compared to direct current (DC) power consumption by an amplifier driven without a control system 212_1. The control system 212_1 can improve linearity of an amplifier, help in automatic calibration of an amplifier over temperature, voltage, and process variations, and/or autocalibration of a phased array system.

The electronic processing system 524 can comprise a hardware electronic processing system that is configured to execute instructions stored in the memory which will cause the control system 212_1 to perform a variety of functions including, but not limited to, turning on/off or reduce voltages/currents provided to various terminals of an amplifier in response to detecting that the signal to be amplified is turned on/off or sensing individual amplifier current values and change the values of different voltages and currents in response to the deviations of the sensed current values from target values.

The input/output system 532 can be configured to provide wired/wireless connection with external devices and systems. For example, the input/output system 532 can comprise an Ethernet port (e.g., a Gigabit Ethernet (GbE) connector) that provides connection to the electronic processing system 206 and/or a router, one or more connectors that provide connection to the RF signal generator 208, a connector that provides connection with an external power supply, a plurality of connectors that provide voltages/currents to one or more amplifiers, a plurality of connectors that can receive voltage/current information from the one or more amplifiers, and connectors that provide connection with a user interface (e.g., a display device). In various implementations, the input/output system 532 can comprise a command and control link to receive messages from the RF signal generator 208 and/or electronic processing system 206.

The input/output system 532 can be configured to receive as input, a signal/trigger/information from the RF signal generator 208 and use the information from this input to determine the voltages and current for an amplifier in the amplifier chain 210_1. As discussed above, the input received from the RF signal generator can be a trigger that conveys information that the RF signal will be turning on in a short while and causes the control system 212_1 to start the power sequencing process and provide appropriate voltages and/or currents to bias the amplifiers in the corresponding amplifier chain 210_1 prior to the arrival of the RF signal. For example, the input from the RF signal generator can be a pulse enable signal which is high when the RF signal is on and low when the RF signal is off. In various implementations, the input from the RF signal generator 208 can be representative of the waveform being output by the DAC 516_1 of the RF signal generator 208. In some implementations, the input can include instructions and/or settings to power on the control system 212_1, to power up an amplifier in the amplifier chain 210_1, and other data to operate the control system 212_1 and an amplifier in the amplifier chain 210_1.

The input/output system 532 can comprise a communication system configured to communicate with external devices and systems. For example, the input/output system 532 can comprise Ethernet connectivity to send information including but not limited to amplifier health information, and efficiency statistics to the electronic processing system 206. Ethernet connectivity can also help in synchronizing an array of many control systems in phased array applications. The input/output system 532 comprises a plurality of connectors that are configured to provide voltages/currents to at least one terminal of an amplifier in the amplifier chain 210_1. For example, the voltages and currents required to bias at least one of the gates, source and/or drain terminal of an amplifier in the amplifier chain 210_1 can be provided through the output ports of the control system 212_1. The control system 212_1 can be configured to provide bias voltage and/or current to a plurality of amplifiers. For example, the control system 212_1 can be configured to provide bias voltage and/or current to two, four, six or more amplifiers.

The sensing system 528 can be configured to sense current values at one or more terminals of the amplifier as discussed above. In various implementations, the sensing system 528 comprises at least one current sensor and an analog to digital converter (ADC) configured to sample and average the output of the current sensor to obtain a sensed current value. In another implementation of the sensing system 528, the voltage drops across a resistor (e.g., a shunt resistor) connected to the drain terminal is measured. The drain current is obtained from the measured voltage drop and the value of the resistor. In such an implementation, the sensing circuit is designed to have low offset voltage and low noise which allows for greater accuracy in the measurement of the drain current. In various implementations, the current sensor need not be integrated with the other components of the sensing system 528 and/or the other sub-systems of the control system 212_1. Instead, the current sensor can be integrated with the amplifier. The number of current sensors can vary based on the number of amplifiers being controlled by the control system 212_1 and the number of currents that are being monitored. For example, if the control system 212_1 is configured to control four distinct amplifiers and it is desired to monitor the drain current of each of the four separate amplifiers, then the control system 212_1 comprises four current sensors configured to monitor the drain current of each of the four distinct amplifiers.

The power adapting system 530 can be configured to convert power from an external power supply 534 (e.g., an AC power line, a battery source, a generator, etc.) to voltage and current waveforms required for operating the amplifiers being controlled by the control system 212_1. For example, in various implementations, the power adapting system 530 is configured to convert a 60V DC bus and generate appropriate voltage and current inputs for the various terminals of the amplifier. In some implementations, the power adapting system 530 may be configured to convert an incoming AC power line to DC power (e.g., DC voltages between about +20 Volts DC and about +80 Volts DC). The power adapting system 530 is configured to step up/down the converted DC voltage to appropriate voltages for the amplifier (e.g., in a voltage range between about +45 Volts and +70 Volts high voltage Gallium Nitride power amplifiers) through DC/DC converters. The stepped up/down voltages are provided to the various terminals of the amplifier (e.g., gate, drain, and/or source) in a sequence as discussed above in response to receiving a signal from the RF signal generator 208 and/or the electronic processing system 206 that the signal to be amplified is turned on/being turned on.

In various implementations, the control system 212_1 comprises a “power gating” feature where the bias voltage/current at various terminals (e.g., gate and/or drain) of the amplifier is adjusted in response to a sensed characteristic of the system and/or target material. In various implementations, the control system 212_1 can provide offset voltages that raise and lower the biasing voltage to turn on/turn off the power amplifier in response to the turning on and turning off the RF signal. For example, in an implementation of the amplifier chain 210_1 comprising a GaN power amplifier, the control system 212_1 can toggle the gate voltage between about-5V (pinch off or turn off) and about −2.5V (saturation or turn on) at a frequency greater than or equal to about 1 kHz and less than or equal to about 500 MHz. As another example, the gate voltage can be toggled between pinch off and saturation at a rate greater than or equal to about 10 MHz and less than or equal to about 100 MHz. Without any loss of generality, the control system 212_1 can be configured to turn-on and turn-off the amplifier in between pulses of a pulsed waveform. This can advantageously allow heat to dissipate from the amplifier in between pulses thereby reducing the rate at which the amplifier heats up and increase lifetime. Turning on and off the amplifier in between pulses of a pulsed waveform can also advantageously increase the power efficiency of the amplifier.

The control system 526 can be configured to control and/or manage various functions and processes of the control system 212_1. For example, the control system 526 independently or in co-operation with the electronic processing system 206 and/or the RF signal generator 208 can control the order in which the voltage and current levels at various terminals of the amplifier are changed to power up/down the amplifier. As another example, the control system 526 independently or in co-operation with the electronic processing system 206 and/or the RF signal generator 208 can control the raising and lowering of the voltage/current levels at the gate terminal of the amplifier synchronously with the incoming signal to be amplified. Yet another example, the control system 526 independently or in co-operation with the electronic processing system 206 and/or the RF signal generator 208 can control the timing of turning on the various amplifiers in the amplifier chains 210_1 to 210_N.

As discussed above, the control system 212_1 can be configured to use the information about the signal to be amplified to adjust/tune bias voltages and currents that power up/down one or more amplifiers in the amplifier chain 210_1 to improve various figures of merit (e.g., power efficiency, linearity, etc.). FIG. 5C is a schematic illustration of an amplifier 536 in the amplifier chain 210_1 that is being controlled by the control system 212_1. The amplifier 536 is a FET amplifier having a gate terminal 538 and a drain terminal 540. As discussed above, the control system 212_1 is configured to provide voltage/current to the gate terminal 538 and the drain terminal 540 of the amplifier as well as adjust the voltage/current levels at the gate terminal 538 and the drain terminal 540 based on information regarding the incoming signal and/or information regarding the temperature and other physical characteristics of the amplifier 536.

The signal to be amplified can be input to the gate terminal 538 via an input matching circuit 542. The amplified signal can be output from the drain terminal 540 via an output matching circuit 544. To ease the burden on the power adapting system 530, one or more storage capacitors 546 are placed near the drain terminal 540 of the amplifier 536. The illustrated implementation comprises a single storage capacitor 546. The one or more storage capacitors 546 can have a capacitance value between about 700 microfarads and 2000 microfarads. The presence of the storage capacitors 546 are advantageous in high power applications and/or applications in which the signal has a high duty cycle. In implementations comprising a plurality of capacitors, the plurality of capacitors can be arranged in parallel. As discussed above, the control system 212_1 comprises a plurality of current sensors 548 and 550 that are configured to sense/monitor drain and gate current respectively. The current sensor 548 configured to monitor/sense drain current can be positioned downstream of the storage capacitor 546 as shown in the illustrated embodiment or upstream of the storage capacitor 546 in other embodiments. As discussed above, the control system 212_1 can also comprise a temperature sensor 552 configured to sense/monitor the ambient temperature in the vicinity of the amplifier 536. For example, the temperature sensor 552 can be configured to measure the temperature of the circuit board on which the amplifier 536 is mounted.

In various implementations, the control system 212_1 can be configured to protect the amplifiers from damage. The control system 212_1 can be configured to monitor voltages and/or currents at various terminals of the amplifier and turn-off the amplifier if the current and/or voltage at one or more terminals of the amplifier exceeds a certain limit. For example, the control system 212_1 can be configured to turn off an amplifier in the amplifier chain 210_1 if the drain current of that amplifier exceeds a preset threshold. The threshold drain current for the various amplifiers controlled by the control system 212_1 can be programmed and stored in a memory accessible by the control system 212_1. The threshold drain current can be different when the RF signal is on and off. As another example, the control system 212_1 is configured to turn-off the amplifier if the rate of increase of the drain current of an amplifier during power up sequence is below a threshold rate. The threshold rate of increase of the drain current for the various amplifiers controlled by the control system 212_1 can be programmed and stored in a memory accessible by the control system 212_1. In various implementations, the control system 212_1 can be configured to monitor the duration of time an amplifier is on and turn off the amplifier if an amplifier is on for an amount time greater than a preset amount of time even if the RF signal is on. The preset amount of time can be programmed and stored in a memory accessible by the control system 212_1. In various implementations, an input switch can be provided in the input signal path of the amplifier. In such implementations, the control system 212_1 can be configured to open the input switch and disconnect the RF signal from the input to the amplifier if the voltage, current and/or duration of time the amplifier is on exceeds a limit. In various implementations, a load switch can be provided in the drain path of the amplifier. In such implementations, the load switch can be opened to disconnect the drain and prevent damage to the amplifier if the drain current exceeds a limit.

The bias voltage/current of an amplifier (e.g., a GaN power amplifier) that optimizes the power efficiency of amplifier can vary based on the device temperature. Thus, the power efficiency of an amplifier can degrade from an optimum power efficiency as the temperature of the amplifier changes. Without relying on any particular theory, the temperature of the amplifier can increase over the duration of time that the amplifier is in use. Thus, it is advantageous to intermittently obtain a measurement/estimate of the temperature of the amplifier during use and adjust the bias voltage/current to optimize power efficiency and/or other figures of merit of the amplifier. The bias voltage/current that optimizes power efficiency can also be affected due to degradation in the device performance due to defects during manufacturing, aging or a defect in the circuitry surrounding the amplifier.

While the temperature sensor 552 in FIG. 5C may provide information regarding the ambient temperature around the amplifier 536. In many implementations, it may not be practical to use a temperature sensor to obtain an estimate of the device temperature of the amplifier 536. However, the drain current can be correlated to the device temperature of the amplifier 536 and can be used to measure the device temperature of the amplifier 536. The variation of the drain current versus temperature can be different when the biasing gate voltage is changed.

The drain current can also provide an indication of a degradation in the performance of the amplifier 536 as a result of defects due to manufacturing/aging or a defect in the circuitry surrounding the amplifier. Thus, adjusting the biasing voltages/currents based on measuring the drain current can advantageously aid in optimizing power efficiency and other figures of merit of the amplifier 536. The drain current can be obtained under bias condition when the signal to be amplified is absent, when the signal to be amplified is present and/or in between signal pulses. For example, in some implementations, the current sensor 548 can be configured to sense the drain current continuously or almost continuously. As discussed above, analog-to-digital converters in the control system 212_1 sample the sensed current. A measurement of the drain current is obtained by averaging over a plurality of samples of the sensed current. The electronic processing system 524 can be configured to correlate the measured drain current to the device temperature of the amplifier 536. The electronic processing system 524 can be configured to correlate the measured drain current to the device temperature of the amplifier 536 using algorithms and/or look-up-tables (LUTs).

As the device temperature of the amplifier 536 changes, the biasing gate voltage that would achieve power efficient operation can change. Accordingly, in many implementations, the electronic processing system 524 of the control system 212_1 can be further configured to change the biasing gate voltage based on the device temperature obtained from the measured gate current. The electronic processing system 524 can be configured to obtain the amount by which the gate voltage should be changed (also referred to herein as gate offset voltage) using algorithms and/or look-up-tables (LUTs). The gate offset voltage can be in a range between about 1 mV and about 500 mV. In various implementations, the signal to be amplified is turned off before changing the gate voltage by the offset amount. In some implementations wherein the signal to be amplified comprises pulses, the gate voltage is changed by the offset amount in the time interval between pulses. In some implementations, the gate voltage is changed by the offset amount when the signal to be amplified is on.

In addition to optimizing power efficiency based on device temperature and/or achieving a desired power efficiency at different temperatures, the control system 212_1 can also help in preventing a rapid increase in the device temperature by adjusting the gate bias voltage as the drain current changes to maintain an optimal gain and/or power efficiency.

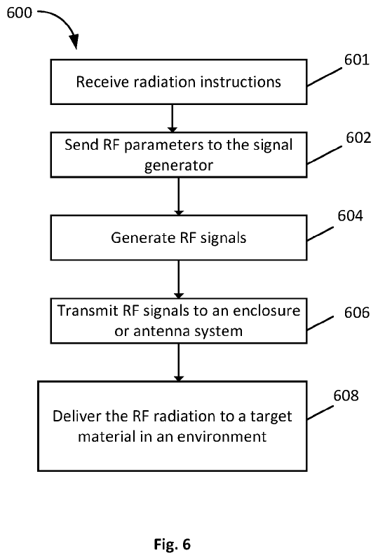

FIG. 6 illustrates a method 600 performed by the RF system 100 and/or 110. The RF system comprises an electronic processing system 206 that executes stored instructions causing the RF system to perform the method 600. At 601, the method 600 includes receiving instructions for generating or emitting EM radiation. The instructions may depend on various operations to be performed by the RF system. For example, sanitization operation of a surface or sterilization operation to a surgical instrument or performing healing operation to a wound or performing sanitization in an environment (or air), etc. At 602, the method 600 includes determining parameters for generating the RF signals and sending those parameters to an RF signal generator. The parameters may include power information, type of waveform, duty cycle, frequency of RF wave, timing of signal, pulse width, etc. At 604, the method 600 includes generating RF signals. At 606, the method includes transmitting RF signals to an enclosure or an antenna system. At 608, the methods include, delivering the RF radiations to a target material in an environment.

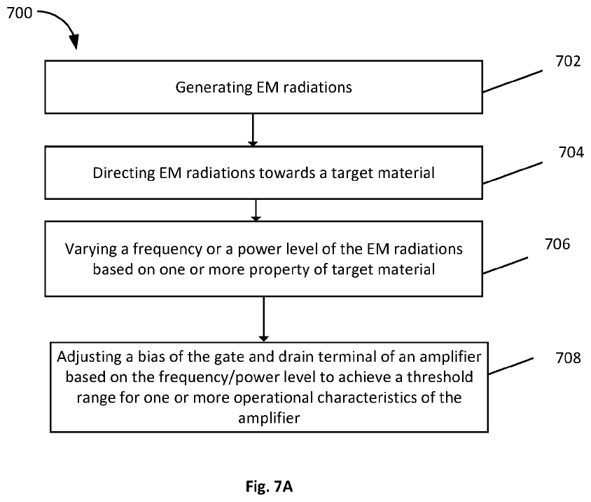

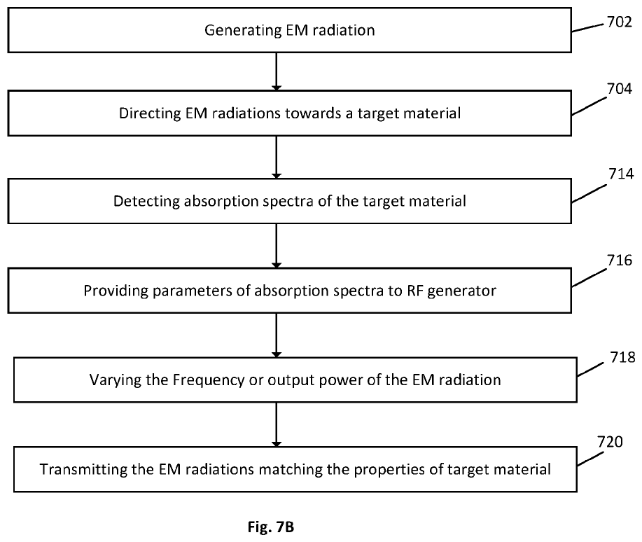

FIG. 7A illustrates a method 700 performed by RF system 100 and/or 110 in accordance with certain aspects of the present disclosure. The RF system comprises an electronic processing system that executes stored instructions causing the RF system to perform the method 700. At 702, the method 700 includes generating EM radiations. At 704, the method includes directing the EM radiation towards a target material. The target material can be virus, bacteria, fungi, parasites, microbes, biological cells, proteins, RNA, and molecules. At 706, the method 700 includes varying a frequency or a power level of EM radiations based on one or more property of target material. For example, the frequency and power levels of EM radiations are varied to address various types of target material present in a volume or environment or surface. At 708, the method 700 includes adjusting a bias of the gate and drain terminal of an amplifier based on the frequency and/or power level to achieve a threshold range for one or more operational characteristics of the amplifier. For example, based on the type of target material and/or the property of the target material, the control circuit adjusts the bias voltage or current of the gate and drain terminal of the amplifying system or one or more amplifier in the amplifying system to achieve efficient deactivation or destruction of the target material.

FIG. 7B illustrates another method performed by RF system 100 and/or 110, in accordance with certain aspects of the present disclosure.

At 714, the method includes detecting the absorption spectra of the target material. For example, when the EM radiation received by the target material, a part of EM radiation is absorbed and transmitted. The absorbed and transmitted radiation information is received by a receiver system (e.g., receiver system 302). The receiver system can comprise a RF detector, processing electronics and an electronic memory. In some implementations, the receiver system may comprise a sensor system configured to send EM radiation scattered by or reflected from the target material. In some implementations, the receiver system can comprise an antenna system configured to receive the radiation scattered by or reflected from the target material. In some implementations, the receiver system can be configured as a spectroscope/spectrometer. As such, the receiver system is configured to detect EM radiation transmitted through the target material or scattered by the target material. At 716, the method includes providing parameters of absorption spectra to the RF generator. For example, the processing electronics associated with the receiver system can use one or more software program to extract the one or more parameters from the absorption spectra and provide the extracted parameters as an input to the RF generator.

Based on the extracted radiation parameters received at 716, the frequency, power and/or other parameters of the EM radiation output from the RF generator are varied at 718. For example, based on the radiation parameters received from receiver system, the frequency and/or the output power of transmitted radiation can be varied to efficiently deactivate or destroy a component of the target material. Additionally, radiation parameters are stored in one or more memory or databases system for any future reference. In one or more implementations, the one or more electronic processing system can refer a look up table or pre-stored information to dynamically generate the radiation parameters to efficiently deactivate or destroy a component of the target material. At 720, the method includes transmitting the EM radiations matching the properties of the target material.

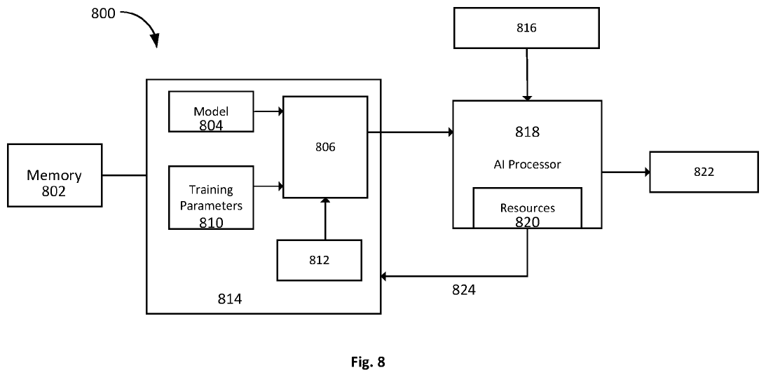

FIG. 8 illustrates a system 800 for training an artificial neural network according to one or more embodiments. The system 800, more particularly, is configured to train a base neural network (NN) model 804 into a trained NN model 822 using a set of training data 816. The training data 816 may include absorption spectrums of various biological target materials described herein, such as viruses, bacteria, prions, and amoebae, by way of non-limiting example. The training data 816 may include data regarding the efficacy of various waveforms (e.g., sine wave, square wave, sawtooth wave) on corresponding types of harmful biological target materials.

The training data 816 may include data regarding time periods of EM radiation emission on corresponding types of biological materials. More specifically, mechanisms described herein effectively neutralize target biological materials by resonating, polarizing, or causing the dielectric breakdown of one or more structures of the target biological materials. Those skilled in the art will appreciate that target biological materials may be effectively neutralized or destroyed according to these mechanisms without increasing the temperature beyond a threshold temperature. For instance, the threshold temperature of the COVID-19 virus is approximately 150° F. Subjecting the COVID-19 virus to EM radiation in frequency ranges other than the resonant frequency for a significant amount of time will cause the COVID-19 virus to reach the critical temperature, thereby destroying the virus. However, this may also increase the temperature of and cause damage to surrounding non-target material (e.g., skin, blood, muscle) of humans or animals. By limiting the time period in which target biological material is subjected to the EM radiation in a resonant frequency range, the target biological material can be neutralized without damaging surrounding tissue.

The training data regarding time periods of EM radiation emission may include data regarding effective time periods for neutralizing types of target material. The training data regarding time periods of EM radiation emission may include data regarding cool down time periods after the effective time periods for allowing types of target material to return to a threshold temperature below the critical temperature. The training data regarding time periods of EM radiation emission may include data regarding safe EM radiation exposure by humans or animals in correlation with various characteristics associated with the EM radiation, such as distance to the EM radiation source, power level and/or frequency of the EM radiation emitted, and/or type of waveform. A trained NN model 822 trained using such data may be implemented in an RF system described herein to automatically neutralize target material(s) without significant human intervention.

The training data 816 may include data regarding operation of the RF transmitter 102 according to the principles described with respect to FIGS. 5A through 5C. For instance, the training data 816 may include data regarding selection of a type of waveform, data regarding power monitoring, data regarding power management, data regarding operation of the RF signal generator 208, and/or data regarding the amplifying system 210. As a more specific yet non-limiting example, the training data 816 may include data indicating bias voltage and/or frequency of one or more terminals of an amplifier correlated with frequency or output power of the EM radiation to be emitted. Therefore, the trained NN model 822 may operate the RF transmitter 102 according to one or more desired operational characteristics, such as efficiency, temperature, or power consumption, by way of non-limiting example.

In this example, one or more electronic processing system(s) 814 may be in communication with one or more AI electronic processing system(s) 818. Electronic processing system(s) 814 may include traditional CPUs, FPGAs, systems on a chip (SoC), ASICs, or embedded ARM controllers, for example, or other electronic processing systems that can execute software and communicate with AI electronic processing system(s) 818 based on instructions in the software. AI electronic processing system(s) 818 may include graphics electronic processing systems (GPUs), AI accelerators, or other digital electronic processing systems optimized for AI operations (e.g., matrix multiplications versus Von Neuman Architecture electronic processing systems such as the x86 electronic processing system). Example AI electronic processing system(s) may include GPUs (e.g., Nvidia Volta® with 800 cores and 64 Multi Accumulators) or a Tensor Electronic processing system Unit (TPU) (e.g., 4 cores with 16 k operations in parallel), for example.

In this example, a control electronic processing system 814 may be coupled to memory 802 (e.g., one or more non-transitory computer readable storage media) having stored thereon program code executable by control electronic processing system 814. The control electronic processing system 814 receives (e.g., loads) a neural network model 804 (hereinafter, “model”) and a plurality of training parameters 810 for training the model 804. The model 804 may comprise, for example, a graph defining multiple layers of a neural network with nodes in the layers connected to nodes in other layers and with connections between nodes being associated with trainable weights. The training parameters 810 (e.g., tuning parameters, model parameters) may comprise one or more values which may be adjusted to affect configuration and/or execution of the model 804. The training parameters 810 that may be used in various embodiments include model size, batch size, learning rate, precision (e.g., number of bits in a binary representation of data values, type of numerical data), sparsity (e.g., number of zero values relative to non-zero values in matrices), normalization (e.g., weight decay, activation decay, L2 normalization), entropy, and/or training steps, by way of non-limiting example. Other parameters or attributes may be included in the training parameters 810 that may be characterized and adjusted as would be apparent to those skilled in the art in light of the present disclosure. In some embodiments, the training parameters 810 may include one or more hyperparameters (e.g., parameters used to control learning of the neural network) as known to those skilled in the art. The electronic processing system 814 may also execute a neural network compiler 806. The neural network compiler 806 may comprise a program that, when executed, may receive model 804 and training parameters 810 and configure resources 820 on one or more AI electronic processing systems 818 to implement and execute model 804 in hardware. For instance, the neural network compiler 806 may receive and configure the model 804 based on one or more of the training parameters 810 to execute a training process executed on AI electronic processing system(s) 818. The neural network compiler 806 may cause the one or more AI electronic processing systems 818 to implement calculations of input activations, weights, biases, backpropagation, etc., to perform the training process. The AI electronic processing system(s) 818, in turn, may use resources 820, as determined by the neural network compiler 806, to receive and process training data 816 with model 804 (e.g., the training process). The resources 820 may include, for example, registers, multipliers, adders, buffers, and other digital blocks used to perform operations to implement model 804. The AI electronic processing system(s) 818 may perform numerous matrix multiplication calculations in a forward pass, compare outputs against known outputs for subsets of training data 816, and perform further matrix multiplication calculations in a backward pass to determine updates to various neural network training parameters, such as gradients, biases, and weights. This process may continue through multiple iterations as the training data 816 is processed. In some embodiments, AI electronic processing system(s) 818 may determine the weight updates according to a backpropagation algorithm that may be configured by the neural network compiler 806. Such backpropagation algorithms include stochastic gradient descent (SGD), Adaptive Moment Estimation (ADAM), and other algorithms known to those skilled in the art.

During training of the model 804, one or more values for activations, biases, weights, gradients, or other parameter may be generated or updated for one or more layers, nodes, and/or connections of the model 804. During training, the AI electronic processing system(s) 818 may generate training information 824 that is useable to determine a status or a progress of training the model 804. The AI electronic processing system(s) 818 may provide the training information 824 to the control electronic processing system(s) 814. The AI electronic processing system(s) 818 and/or the control electronic processing system 814 may use the training information 824 to determine whether to adjust various parameters or attributes of the neural network training process. The control electronic processing system 814 may obtain or possess training criteria 812 for determining whether to adjust the training attributes or parameters.

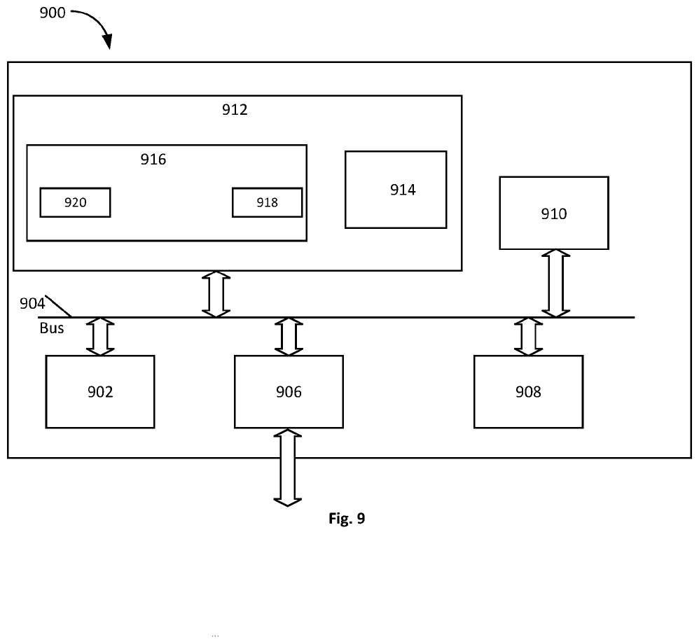

FIG. 9 illustrates a simplified block diagram of an example computer system according to various embodiments.

The features described herein may include one or more components of a computer system 900. Computer system 900 can be used to implement any of the computing devices, systems, or servers described in the foregoing disclosure. As shown in FIG. 9, computer system 900 includes one or more electronic processing systems 902 that communicate with a number of peripheral devices via a bus subsystem 904. These peripheral devices include a storage subsystem 912 (comprising a memory subsystem 916 and a file storage subsystem 914), user interface input devices 910, user interface output devices 908, and a network interface subsystem 906.

Bus subsystem 904 can provide a mechanism for letting the various components and subsystems of computer system 900 communicate with each other as intended. Although bus subsystem 904 is shown schematically as a single bus, alternative embodiments of the bus subsystem can utilize multiple busses.

Network interface subsystem 906 can serve as an interface for communicating data between computer system 900 and other computer systems or networks. Embodiments of network interface subsystem 906 can include, e.g., an Ethernet card, a Wi-Fi and/or cellular adapter, a modem (telephone, satellite, cable, ISDN, etc.), digital subscriber line (DSL) units, and/or the like.

User interface input devices 910 can include a keyboard, pointing devices (e.g., mouse, trackball, touch-pad, etc.), a touchscreen incorporated into a display, audio input devices (e.g., voice recognition systems, microphones, etc.) and other types of input devices. In general, use of the term “input device” is intended to include all possible types of devices and mechanisms for inputting information into computer system 900.

User interface output devices 908 can include a display subsystem, a printer, or non-visual displays such as audio output devices, etc. The display subsystem can be, e.g., a flat-panel device such as a liquid crystal display (LCD) or organic light-emitting diode (OLED) display. In general, use of the term “output device” is intended to include all possible types of devices and mechanisms for outputting information from computer system 900. Storage subsystem 912 includes a memory sub-system 916 and a file storage subsystem 914. Sub-systems 916 and 914 represent non-transitory computer-readable storage media that can store program code and/or data that provide the functionality of embodiments of the present disclosure.

Memory subsystem 916 includes a number of memories including a main random-access memory (RAM) 918 for storage of instructions and data during program execution and a read-only memory (ROM) 920 in which fixed instructions are stored. File storage subsystem 914 can provide persistent (i.e., non-volatile) storage for program and data files, and can include a magnetic or solid-state hard disk drive, an optical drive along with associated removable media (e.g., CD-ROM, DVD, Blu-Ray, etc.), a removable flash memory-based drive or card, and/or other types of storage media known in the art.

It should be appreciated that computer system 900 is illustrative and many other configurations having more or fewer components than system 900 are possible.

### OTHER VARIATIONS

Features, materials, characteristics, or groups described in conjunction with a particular aspect, embodiment, or example are to be understood to be applicable to any other aspect, embodiment or example described herein unless incompatible therewith. All the features disclosed in this specification (including any accompanying claims, abstract and drawings), and/or all the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only and are not intended to limit the scope of protection. Indeed, the novel methods and systems described herein may be embodied in a variety of other forms. Furthermore, various omissions, substitutions, and changes in the form of the methods and systems described herein may be made. Those skilled in the art will appreciate that in some embodiments, the actual steps taken in the processes disclosed and/or illustrated may differ from those shown in the figures. Depending on the embodiment, certain of the steps described above may be removed, others may be added. For example, the actual steps and/or order of steps taken in the disclosed processes may differ from those described and/or shown in the figure. Depending on the embodiment, certain of the steps described above may be removed, others may be added. For instance, the various components illustrated in the figures and/or described may be implemented as software and/or firmware on a electronic processing system, controller, ASIC, FPGA, and/or dedicated hardware. Furthermore, the features and attributes of the specific embodiments disclosed above may be combined in different ways to form additional embodiments, all of which fall within the scope of the present disclosure.

In some cases, there is provided a non-transitory computer readable medium storing instructions, which when executed by at least one computing or processing device, cause performing any of the methods as generally shown or described herein and equivalents thereof.

Any of the memory components described herein can include volatile memory, such random-access memory (RAM), dynamic random access memory (DRAM), synchronous dynamic random access memory (SDRAM), double data rate (DDR) memory, static random access memory (SRAM), other volatile memory, or any combination thereof. Any of the memory components described herein can include non-volatile memory, such as magnetic storage, flash integrated circuits, read only memory (ROM), Chalcogenide random access memory (C-RAM), Phase Change Memory (PC-RAM or PRAM), Programmable Metallization Cell RAM (PMC-RAM or PMCm), Ovonic Unified Memory (OUM), Resistance RAM (RRAM), NAND memory (e.g., single-level cell (SLC) memory, multi-level cell (MLC) memory, or any combination thereof), NOR memory, EEPROM, Ferroelectric Memory (FeRAM), Magnetoresistive RAM (MRAM), other discrete NVM (non-volatile memory) chips, or any combination thereof.

Any user interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, or within less than 0.01% of the stated amount.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

The foregoing description, for purposes of explanation, used specific nomenclature to provide a thorough understanding of the disclosure. However, it will be apparent to one skilled in the art that specific details are not required in order to practice the disclosed embodiments. Thus, the foregoing descriptions of specific embodiments are presented for purposes of illustration and description. They are not intended to be exhaustive or to limit the disclosure to the precise forms disclosed; obviously, many modifications and variations are possible in view of the above teachings. The embodiments were chosen and described in order to best explain the principles of the disclosure and its practical applications, they thereby enable others skilled in the art to best utilize the disclosure and various embodiments with various modifications as are suited to the particular use contemplated. It is intended that the claims as presented herein or as presented in the future and their equivalents define the scope of the protection.

1. A system, comprising:
a transmitter configured to generate electromagnetic (EM) radiation characterized by a frequency and an output power and direct the generated EM radiation towards a target organic material in a volume, the transmitter including an amplifier having a gate terminal and a drain terminal, wherein the frequency or the output power of the generated EM radiation is selected based on a type or a property of the target organic material; and
a control system configured to adjust a bias voltage or a bias current provided to the gate terminal or the drain terminal of the amplifier based on the frequency or the output power.

2. The system of claim 1, wherein the target organic material is selected from a group consisting of virus, bacteria, fungi, parasites, microbes, biological cells, proteins, RNA, and molecules.

3. The system of claim 1, wherein the frequency of the generated EM radiation is resonant with a constituent of the target organic material.

4. The system of claim 1, wherein the property of the target organic material comprises at least one property of a physical property, a chemical property, or a surface property.

5. The system of claim 1, further comprising:
a receiver system configured to receive EM radiation transmitted through the target organic material or scattered by the target organic material.

6. The system of claim 5, wherein the output power or the frequency of the EM radiation is dynamically adjusted based on a parameter of the received EM radiation transmitted through the target organic material or scattered by the target organic material.

7. The system of claim 5, wherein the receiver system comprises a radio frequency (RF) detector, processing electronics, and an electronic memory.

8. The system of claim 7, wherein the receiver system is configured to:
compare the received EM radiation with a set of data stored in the electronic memory;
identify, based on the comparison, a type of the target organic material or a property of the target organic material; and
provide information regarding frequency or output power to the transmitter based on the identified type of the target organic material or the property of the target organic material.

9. The system of claim 5, wherein the receiver system comprises a sensor.

10. The system of claim 5, wherein the receiver system comprises a spectrometer.

11. The system of claim 1, wherein the frequency of the generated EM radiation is safe for in-vivo applications.

12. The system of claim 1, wherein the transmitter further comprises a sensor system configured to detect presence of a human or an object in an environment surrounding the system.

13. The system of claim 12, wherein the frequency or the output power of the generated EM radiation is dynamically adjusted based on an output of the sensor system.

14. The system of claim 12, wherein the sensor system comprises at least one sensor of a motion sensor, a temperature sensor, a proximity sensor, or an infrared (IR) camera device.

15. The system of claim 1, comprising a mounting plate configured to mount the system to a surface.

16. The system of claim 1, configured to be handheld.

17. The system of claim 1, wherein the frequency of the generated EM radiation is greater than or equal to 200 MHz and less than or equal to 100 GHz.

18. The system of claim 1, wherein the generated EM radiation is a pulsed waveform comprising a plurality of pulses having a pulse width and a duty cycle.

19. The system of claim 18, wherein the pulse width and the duty cycle is variable.

20. A method, comprising:
generating electromagnetic (EM) radiation characterized by a frequency and an output power, via a transmitter that includes an amplifier having a gate terminal and a drain terminal;
directing the EM radiation toward a target organic material disposed in a volume;
dynamically varying the frequency or the output power of the EM radiation generated based on one or more properties of the target organic material; and
adjusting a bias voltage or a bias current provided to the gate terminal or the drain terminal based on the frequency or the output power.

21-24. (canceled)

25. A non-transitory computer-readable storage medium having instructions stored thereon that, as a result of execution by one or more processing electronics, cause a a radio frequency (RF) system to:
generate electromagnetic (EM) radiation characterized by a frequency and an output power via a transmitter that includes an amplifier having a gate terminal and a drain terminal;
direct the EM radiation toward a target organic material disposed in a volume;
dynamically vary the frequency or the output power of the EM radiation generated based on one or more properties of the target organic material; and
adjust a bias voltage or a bias current provided to the gate terminal or the drain terminal based on the frequency or the output power.

26-29. (canceled)
